(19) United States
(12) Patent Application Publication
(10) Pub. No.: US 2026/0176652 A1
(43) Pub. Date: Jun. 25, 2026

(54) METHODS OF TREATING GENETIC HEARING LOSS

(71) Applicant: UNIVERSITY OF IOWA RESEARCH FOUNDATION, Iowa City, IA (US)

(72) Inventors: Richard J. Smith, Iowa City, IA (US); Paul T. Ranum, Iowa City, IA (US)

(73) Assignee: UNIVERSITY OF IOWA RESEARCH FOUNDATION, Iowa City, IA (US)

(21) Appl. No.: 19/264,470

(22) Filed: Jul. 9, 2025

Related U.S. Application Data
(63) Continuation of application No. 18/968,338, filed on Dec. 4, 2024, now abandoned.
Application No. 18/968,338 is a continuation of application No. 16/635,422, now abandoned.
(60) Provisional application No. 62/540,890, filed on Aug. 3, 2017.

(51) Int. Cl. C12N 15/86; A61K 9/00; A61K 48/00; G01N 33/68

(52) U.S. Cl. CPC C12N 15/86; A61K 48/0066; G01N 33/6893; A61K 9/0019; C12N 2310/141; C12N 2310/20

(57) ABSTRACT

In certain embodiments the present invention provides a method of treating hearing loss comprising: (a) administering a gene suppression agent that suppresses both copies of an endogenous gene causing the hearing loss; and (b) administering an exogenous wild-type allele engineered to resist suppression by the gene suppression agent.

The present invention provides in certain embodiments a method of treating a genetic hearing loss (GHL) in a patient in need thereof comprising: (a) identifying a mutation in a GHL-causing gene, wherein the mutation causes GHL in the patient; and (b) administering to the patient a pharmaceutical composition comprising a therapeutic miRNA and a pharmaceutically acceptable carrier, wherein the GHL therapeutic miRNA is of 18 to 25 nucleotides in length and knocks-down the GHL-causing gene function at a higher level than it knocks-down gene function in a corresponding wild-type gene.

### PRIORITY APPLICATION

This application is a continuation of U.S. patent application Ser. No. 16/635,422 filed Jan. 30, 2020, which is a 35 U.S.C. § 371 application of International Application Serial No. PCT/US2018/044996 filed Aug. 2, 2018, which claims priority to U.S. Provisional Application No. 62/540,890 that was filed on Aug. 3, 2017. The entire content of these applications referenced above are hereby incorporated by reference herein.

### STATEMENT REGARDING FEDERALLY SPONSORED RESEARCH

This invention was made with government support under R01DC003544 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Feb. 9, 2026, is named 17023.214US3 and is 15,219 bytes in size.

### BACKGROUND OF THE INVENTION

Hearing loss affects 15-26% of the world's population. Among the elderly, it ranks as the most common neurological disability, impacting ˜50% of octogenarians, affecting in aggregate 360 million people worldwide. Progressive hearing loss can generally be categorizd as conductive hearing loss, sensorineural hearing loss (SNHL), or mixed hearing loss. Conductive hearing loss occurs when hearing loss is due to problems with the ear canal, ear drum, or middle ear and its bones (the malleus, incus, and stapes). Sensorineural hearing loss (SNHL) occurs when hearing loss is due to problems of the inner ear, also known as nerve-related hearing loss. Mixed hearing loss refers to a combination of conductive and sensorineural hearing loss, where there may be damage in the outer or middle ear and in the inner ear (cochlea) or auditory nerve.

“Nonsyndromic deafness” is hearing loss that is not associated with other signs and symptoms. In contrast, “syndromic deafness” involves hearing loss that occurs with abnormalities in other parts of the body. Different types of nonsyndromic deafness are generally named according to their inheritance patterns. Nonsyndromic deafness can occur at any age. About 1 in 1,000 children in the United States is born with profound deafness, and another 2 to 3 per 1,000 children are born with partial hearing loss. More than half of these cases are caused by genetic factors. Most cases of genetic deafness (70 to 80%) are nonsyndromic and the remaining cases are caused by specific genetic syndromes.

Researchers have identified more than 90 genes that, when altered, are associated with nonsyndromic deafness; however, some of these genes have not been fully characterized. Many genes related to deafness are involved in the development and function of the inner ear. Mutations in these genes contribute to hearing loss by interfering with critical steps in processing sound. Different mutations in the same gene can be associated with different types of hearing loss, and some genes are associated with both syndromic and nonsyndromic deafness. Nonsyndromic deafness can have different patterns of inheritance. 20% to 25% of nonsyndromic deafness cases are autosomal dominant (i.e., one copy of the altered gene in each cell is sufficient to result in hearing loss). Genetic testing for hearing loss provides important information for patients such as prognosis and treatment options, and genetic counseling.

Current treatments for hearing loss include the use of hearing aids, cochlear implants and brainstem implants. Both hearing aids and cochlear implants amplify sounds to enable deaf people to hear, to distinguish environmental sounds and warning signals, and to modulate the voice and make speech more intelligible. Brain stem implants help persons who have had both acoustic nerves destroyed (e.g., by bilateral temporal bone fractures or neurofibromatosis) have some sound perception restored by means of electrodes connected to from sound-detecting and sound-processing devices directly to the brain stem. Genetic hearing loss is highly heterogeneous, as hundreds of mutations in the more than 90 genes have been discovered as causing hearing loss. This makes genetic testing for hearing loss difficult using traditional DNA (Sanger) sequencing methods, which rely on sequencing a single gene at a time.

Currently, there is a need for effective treatments to mitigate genetic hearing loss.

### SUMMARY OF THE INVENTION

In certain embodiments, the present invention provides a method of treating hearing loss comprising: (a) administering a gene suppression agent that suppresses both copies of an endogenous gene causing the hearing loss; and (b) administering an exogenous wild-type allele engineered to resist suppression by the gene suppression agent.

In certain embodiments, the present invention provides a method of treating genetic hearing loss (GHL) in a patient in need thereof comprising administering to a patient identified as having a mutation in a GHL-causing gene a pharmaceutical composition comprising pharmaceutically acceptable carrier and a GHL therapeutic miRNA, wherein the miRNA is of 18 to 25 nucleotides in length and suppresses expression of the GHL-causing gene to a greater level than it suppresses expression of a corresponding wild-type gene, wherein the GHL-causing gene is an exon listed in Table 1.

In certain embodiments, the present invention provides a method of treating a genetic hearing loss (GHL) in a patient in need thereof comprising: (a) identifying a mutation in a GHL-causing gene, wherein the mutation causes GHL in the patient, and wherein the GHL-causing gene is an exon listed in Table 1; and (b) administering to the patient a pharmaceutical composition comprising a therapeutic miRNA and a pharmaceutically acceptable carrier, wherein the GHL therapeutic miRNA is of 18 to 25 nucleotides in length aFnd knocks-down the GHL-causing gene function at a higher level than it knocks-down gene function in a corresponding wild-type gene.

In certain embodiments, the present invention provides a method of transducing cochlear epithelial tissue in an animal, comprising administering rAAV comprising a therapeutic agent to the animal, wherein the administration is intravenously and the rAAV crosses the blood-labyrinthine barrier in the animal, and wherein the rAAV2/9 transfects spiral ganglion neurons, inner hair cells, outer hair cells, stria vascularis, and/or vestibular organs.

In certain embodiments, the present invention provides a method of treating hearing loss in a patient in need thereof, comprising administering a viral vector through a round window membrane of the patient, wherein the patient previously received a canalostomy.

In certain embodiments, the present invention provides a method of transducing cochlear tissue in an animal, comprising: (a) making a post-auricular incision, (b) making a hole with an otologic drill in the chchlea bulla and the posterior semicircular canal, (c) puncturing the RWM, and (d) microinjecting a therapeutic agent into a scala tympani.

In certain embodiments, the present invention provides a method of transducing cochlear tissue in an animal, comprising administering rAAV2/9 intravenously by means of superficial temporal vein to the animal.

In certain embodiments, the present invention provides a method for detecting that a subject is has a gene associated with genetic hearing loss comprising: (a) providing a biological sample from the subject; and (b) contacting the biological sample with at least one first oligonucleotide probe at least 8 nucleotides in length that is complementary to a sequence that comprises an exon listed in Table 1.

### BRIEF DESCRIPTION OF DRAWINGS

FIGS. 1A-1B. Single cell isolation. FIG. 1A. Single cells are identified by morphology. FIG. 1B. During the dissection process, each cell was photographed and catalogued, recording cell type, cochlear region, animal age, time from death to cell harvest, and lysis. All cells were isolated within 30 minutes.

FIGS. 2A-2G. The transcriptional complexity of MYO15A. RNA-Seq identifies the novel alternative transcription start site (FIG. 2B, element “A” depicted in FIG. 2A), the novel mini-exon 8 (FIG. 2C, element “B” depicted in FIG. 2A), and the novel cassette exon 26 with its upstream alternative acceptor site that includes an additional 162 nucleotides (FIG. 2D, element “C” depicted in FIG. 2A). Additional complexity was recognized including an alternative splice acceptor for exon 7 in OHCs (FIG. 2E, element “D” depicted in FIG. 2A), a marked change in transcription at exon 27 observed in all 3 cell types (FIG. 2F, element “E” depicted in FIG. 2A), and alternative splicing between IHCs and OHCs in the 3′ UTR, the significance of which is unknown (FIG. 2G, element “F” depicted in FIG. 2A) (DCs, Deiters Cells; IHCs, inner hair cells; OHCs, outer hair cells).

FIGS. 3A-3D. FIGS. 3A-3C. Single cells are isolated from the cochlea at multiple time points (initially focusing on P15, P60 and P228). FIG. 3D is a single Dieters cell and in path A, a tSNE plot of RNASeq data from 230 individual cells [IHCs, OHCs, Deiters cells]. Path B shows a portion of the transcription complexity of MYO15A. Novel ear-specific exons and gene isoforms are sought in all genes implicated in NSHL. Path C shows pathway analysis on P15 OHCs harvested from Beethoven (Bth) mice (Tmc1Bth/+). The pathway is centered on the RARA gene (Retinoic Acid Receptor Alpha), which is up-regulated in OHCs of Bth mice.

FIGS. 4A-4K. Frequently seen cell types. Shown are many of the additional cell types seen during the single cell procedure. FIGS. 4A and 4B show groups of partially dissociated cells containing OHCs. In FIG. 4A, a large round cell can be clearly seen with a prominent nucleus located in close proximity to the OHCs and several smaller round cells also with prominent nuclei. FIG. 4B shows a wider assortment of round cells at the top with a range of diameters from roughly 10-30 um. The proximity of these cells to the outer hair cells makes it likely that they are either Hensen's or Claudius' cells. FIGS. 4C-D show cells that are less round in shape and more elongated. FIGS. 4E-H show round cells of varying sizes. FIGS. 41-K are round cells isolated from the stria vascularis.

FIGS. 5A-5B. RNAi as a therapy for hearing loss. FIG. 5A. Wild-type vs. Bth Tmc1 mRNA levels. To measure the in vivo effect of the most potent miRNA, we completed allele-specific qRT-PCR on individually isolated hair cells harvested from untreated and treated ears, normalizing all samples to beta actin. The level of wild-type Tmc1 mRNA measured in the untreated ear was set at 1 and the abundance of mRNAs was calculated relative to this sample. Abundance of Tmc1 and Bth Tmc1 was measured in samples containing 12 cells collected from untreated or treated ears from five Beethoven mice at P28. mRNA abundance was calculated using the ΔΔCT method. As can be seen, expression of the Bth allele was suppressed by >88% as compared to levels of Bth mRNA in the untreated ear. The range indicated by the error bars represents the standard deviation of ΔΔCT based on the fold difference calculation 2-ΔΔCt with ΔΔCT+S and ΔΔCT−S. FIG. 5B. Click ABR thresholds in wild-type, untreated Tmc1Bth/+ and treated Tmc1Bth/+ mice. Thresholds in the wild-type mice (˜30 dB SPL) are shown in black. The dashed purple line just above the black line shows thresholds in the two best performing Tmc1Bth/+ mice that received miRNA treatment. The two worst performing animals are shown with the dotted purple line; the solid purple line represents all treated animals (n=10). In the absence of treatment (green line, n=10) or with a scrambled miRNA (orange line, n=13), hearing loss progresses rapidly (two asterisks, p-values <. 005).

FIGS. 6A-6C. Inner ear transduction in adult mice. FIG. 6A. Viral inoculation is performed through the round window membrane as we have described, 7 however a canalostomy is created first to facilitate resistance-free flow through the bony labyrinth. FIG. 6B. Using this method, robust transduction of cochlear tissue is possible in animals of any age. Shown is the transduction efficiency and specificity of Anc80 carrying a CMV-eGFP expression construct delivered to wild-type murine cochlea at P60. Overlapping MYO7A (red) and eGFP (green) localization represent positive hair cell transduction (scale bar, 100 um). FIG. 6C. Note that robust transduction of IHCs is possible without iatrogenic hearing loss. ABRs obtained 3 wks after inoculation show that hearing is preserved (n=3).

FIGS. 7A-7B. Overview of experiments. Older Tmc1Bth/+ mice are treated using the miRNA previously validated in P0-1 Tmc1Bth/+ mice as effective in preserving hearing (FIG. 7A). The experiments initially focus on P15 and P60 Tmc1Bth/+ mice. The goal of this experiment is to determine: a) whether hearing preservation is possible in older animals; 2) if there is a ‘time window’ after the onset of hearing loss during which gene therapy can reverse hearing loss; and, 3) whether there is a time point after which hearing loss is irreversible even with effective suppression of the deafness-causing Bth allele. The miRNA is delivered in Anc-80 using the surgical approach described in FIGS. 6A-6C. The experimental time course is shown and includes allele-specific single cell qRT-PCR 4 wks after surgery, ABRs and DPOAEs every 4 wks during the study, and animal sacrifice with cochlear dissection and immunohistochemistry 35 wks post-surgery. A modification of RNAi-mediated gene therapy using Tmc1Bth/+ and Tmc1Bth/Bthmice as models of ADNSHL and ARNSHL are tested, respectively (FIG. 7B). A new miRNA designed to suppress both endogenous Tmc1 alleles is tested. In the same viral vector, wild-type Tmc1 cDNA that we have engineered to carry synonymous nucleotide changes at the miRNA binding site to prevent binding and miRNA-mediated suppression is packaged.

FIGS. 8A-8D. Inner ear schematic showing established delivery approaches and the systemic approach. Confocal images of representative whole-mount fluorescenceimmunolabeling mouse cochlea and vestibule. (FIG. 8A) Vector delivery into the perilymph via a cochleostomy, canalostomy or trans-round window membrane; vector delivery into the endolymph via cochleostomy of the scala media space; systemic delivery via the superficial temporal vein. (FIG. 8B) Image showing a P0-1 pup after cryoanesthesia under infrared light. The superficial temporal vein for injection is shown in the dotted circle. (FIG. 8C) Representative confocal image of mouse cochlea showing three rows of outer hair cells (OHCs) and one row of inner hair cells (IHCs). (FIG. 8D) Representative confocal image of mouse vestibule showing utricle, anterior semicircular canal (ASCC) and lateral semicircular canal (LSCC). Note that red is phalloidin for labeling of filamentous actin.

FIGS. 9A-9D. Bilateral rAAV2/9 infection via systemic inoculation is dose dependent. Injected mice (high and low dose) were sacrificed 4wks after rAAV2/9 inoculation. Ears were fixed, dissected and stained as cochlear whole mounts. All images were stained with Myo7a (red) for labeling hair cells and imaged for native eGFP (green). (FIG. 9A) 10× images of representative whole-mount apical turns from the higher-titer injected mice showing both ears. There are no differences in eGFP expression. Arrowheads show the pical tip and 8 and 16 kHz regions along the apical turn of the cochlea. (FIG. 9B) 10× images of representative whole-mount apical turns from the lower-titer injected mice. As compared to (a), there is a significant decrease in IHC transduction in the apical turn. (FIG. 9C) 40× magnification at the indicated position in relation to the cochlear apex. IHCs and the three rows of OHCs are shown. (FIG. 9D) The efficiency of rAAV2/9 transduction in IHCs was quantified with 20× images of whole-mount cochlea compiled into cochleograms at 4 weeks. IHCs were counted in 0.25 mm segment and plotted against the distance (%) from the apex. Compared to the lower-titer injection, the higher-titer injection resulted in much stronger rAAV2/9 transduction, with similar transduction in both ears. Data are means±SD (n, number of cochleas; * p<0.05; ** p<0.005).

FIGS. 10A-10F. rAAV2/9 transduction in the spiral ganglion (SG) and organ of Corti 4 weeks after virus inoculation at P0-1. Note that all images were immunostained with anti-GFP (green) antibody and phalloidin (red) to label filamentous actin. (FIG. 10A) Midmodiolar cross-sectional images show transduced SG cells in Rosenthal's canal (RC) (CN, cochlear nerve). (FIG. 10B, 10C) Images in cross-sections of the apical (FIG. 10B) and basal (FIG. 10C) are highmagnification views of the regions marked with white dotted squares. eGFP expression in RC was greater in the apical turn than in the basal turn. (FIG. 10D) In a cross section of confocal image, rAAV2/9 infected the stria vascularis (SV). (FIG. 10E, 10F) High-magnification images show eGFP expression around capillary in SV.

FIGS. 11A-11D. rAAV2/9 transduction in the vestibule 4 weeks after virus inoculation at P0-1. All images, except insets, were stained with Myo7a (red) for labeling hair cells (HCs) and imaged for native eGFP (green). (FIG. 11a-a″) Confocal images of whole mounts of the utricle. eGFP expression is evident throughout the utricle. (FIG. 11b-b″) High-magnification images of the HC layer show transduced HCs (eGFP-positive cells). (FIG. 11c-c″) High magnification images of the SC layer transduced supporting cells (eGFP-positive cells). (FIG. 11d-d″) Confocal images of whole mounts of the Crista ampullaris (CA). eGFP expression is evident throughout the CA. Insets: Cross-sectional images of the utricle and CA. Images were stained with eGFP (green) and phalloidin (red) for labelling hair cells and filamentous actin, respectively. Note hair cell and supporting cell transduction consistent with whole mounts figures.

FIGS. 12A-12B. Comparison of the ABR data from P0-1 injected and control mice. (FIG. 12A) Representative ABR traces recorded from injected (high and low titer) mice 4wks after AAV2/9 inoculation at P0-1 (high and low dose) and the control 4-wk-old mice. Waveforms from injected and control mice appear similar. (FIG. 12B) Click and tone-burst ABRs show relatively normal threshold profiles in the injected mice compared with control mice at 4wks (n, numbers of ears). Slight differences of ABR threshold of less than 10 dB were observed across all frequencies. Data are means+SEM.

FIGS. 13A-13B. Evaluation of the Efficiency and Specificity of AAV Transduction. (FIG. 13A) Comparative transduction efficiency and specificity of rAAV2/1 and rAAV2/9 carrying a CMV-eGFP expression construct delivered to wild-type murine cochlea at P0-1 by intravenous injection. Higher resolution images in the apical turn, utricle and crista mpullaris (CA). Green represents eGFP expression and red is Myo7a for labelling of hair cells. Overlapping Myo7a and eGFP localization represents positive hair cell transduction. (FIG. 13B) The efficiency of viral transuction in IHCs was assessed in 400 μm segments in the apical and basal turns (rAAV2/1 [gray] and rAAV2/9 [black]).

FIGS. 14A-14C. FIG. 14A. Schematic diagram illustrating a variant-level strategy for RNAi-based suppression of a dominant hearing loss mutation (this is the patent we already have). This strategy achieves allele-specific suppression by direct targeting of the causative variant to suppress transcript generation from the mutant copy of the gene without altering transcript levels originating from the endogenous wild type copy. Because this strategy relies on the presence of the wild type copy of the gene, it is suitable for treating autosomal dominant forms of genetic deafness. FIG. 14B. Schematic diagram illustrating the strategy for RNAi-based suppression of both endogenous alleles coupled with replacement with an exogenous engineered wild-type copy resistant to RNAi-based suppression by the therapeutic miRNA. In this strategy, the miRNA binds a position on both copies of the endogenous gene of interest facilitating suppression of both copies of the gene. Simultaneously, an engineered copy of the wild-type gene is delivered and expressed. This gene-level therapeutic strategy can be used to target dominant and recessive forms of genetic deafness. High quality miRNA seed sequences can be selected from across the entire transcript resulting in miRNA constructs with excellent suppression performance and few off-target effects. FIG. 14C. Detailed schematic illustrating the binding of a siRNA (siRNA D11) to mRNA made from both an endogenous and exogenous engineered copy of wild type Tmc1. Binding and suppression are robust when siRNA D11 targets Exon 17 of endogenous wild type Tmc1 but not when siRNA D11 targets the exogenous engineered copy of wild type Tmc1 Exon 17 because of the introduction of synonymous nucleotide variants. These synonymous variants introduce six mismatches at this position the result of which is low binding affinity of siRNA D11. FIG. 14C discloses SEQ ID NOS 1-5, 2, 1, 1, 4, 3, 6, 7, 5, 1, and 8, top to bottom, left to right, respectively, in order of appearance.

FIGS. 15A-15B. FIG. 15A. qPCR results measure the suppression of wild type Tmc when treated with a variety of miRNA designs. miRNA D11 (highlighted with an orange box) performs the best with greater than 80% suppression as compared to the Empty Vector control (far left). Experiments were performed in tissue cultured Cos7 cells, which do not express endogenous Tmc1. Mouse wild type Tmc1 was transfected together with the therapeutic miRNA constructs. Levels of wild type Tmc1 mRNA were measured after 48 hours. qPCR was performed in biological triplicate. Each biological replicate was performed in technical triplicate. Each bar indicates one biological replicate. Error bars indicate standard deviation between technical replicates. FIG. 15B. miRNA are packaged into viral vectors for in vivo delivery. This figure shows the plasmid combinations created, as well as a schematic diagram of the plasmid packaged into AAV2/9 or AAV2/Anc80 capsids. The miRNA construct is driven by the mU6 promoter and the engineered Tmc1 gene is driven by the CMV promoter.

### DETAILED DESCRIPTION OF THE INVENTION

More than 150 genes harboring more than 7000 different genetic mutations have been implicated in non-syndromic hearing loss and the more common forms of syndromic hearing loss. These mutations cause predominantly ARNSHL or ADNHSL, although examples of X-linked and mitochondrial deafness also occur.

The large number of genetic mutations makes the development of mutation-specific gene therapy challenging from an economic and practical perspective. The cost of drug development is likely to be high and the use of any specific mutation-targeting construct may be limited to only a few dozen persons. As an alternative to a mutation-specific approach, our invention is gene-specific therapy. Rather than focusing on a specific mutation in a given gene, we propose to focus on the gene itself. Using this strategy reduces the number of necessary therapeutics from over 7000 for a mutation-specific approach to approximately 150 for a gene-specific approach, making personalized precision medicine for hearing loss practical.

### Single-Cell Dissection and Isolation

The organ of Corti in the inner ear is the receptor apparatus for hearing. It has a large number of highly specialized types of cells including two kinds of hair cells (inner hair cells (IHCs), outer hair cells (OHCs)), five types of supporting cells (Hensen's cells, Deiters' cells, pillar cells, inner phalangeal cells, border cells), three types of cells in the stria vascularis (marginal, intermediate and basal cells), two types of cells in Reissner's membrane (endolymphatic- and perilymphatic-exposed), and four types of fibrocytes (type I-IV). It also includes other cells, the coordinated function of which is to make hearing possible. Testing cells from the organ of Corti is challenging because it is housed in the temporal bone, the densest bone in the body, and some of the cells are quite rare. For example, there are only 3,500 IHCs and 12,000 OHCs, which means that if inner ear tissue is studied without dissecting out individual cells, the genetic signature of any given cell type because masked.

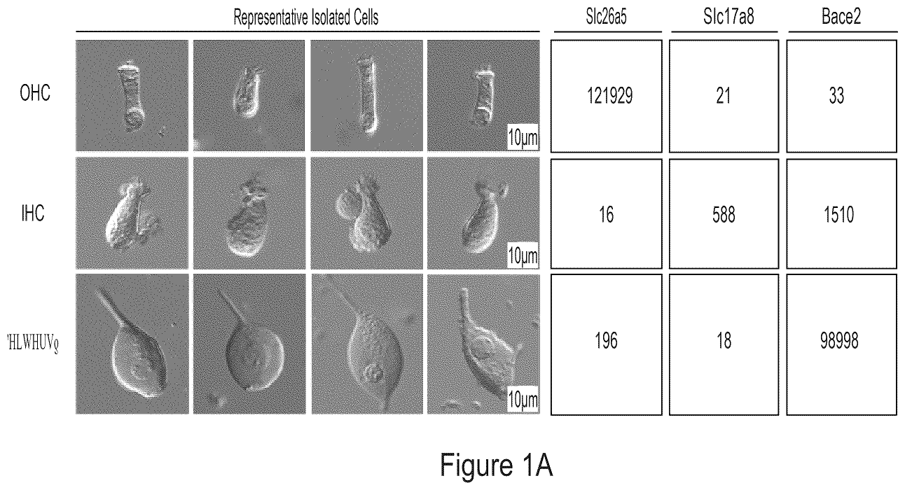

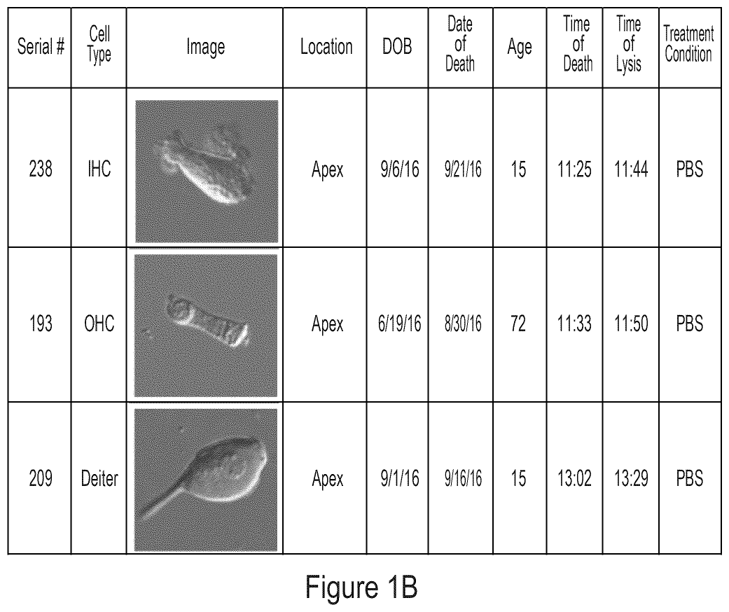

A technique to dissect out individual cells in the inner ear, include IHCs, OHCs and Deiter's cells was perfected. Single cells were identified by morphology (FIGS. 1A-1B). OHCs have an elongated tubular shape and short stereocilia. The nucleus is distinctly visible at the basal end of the cell. IHCs are more flask-like in shape and have distinct indentations that separate the cuticular plate from the cell body. Their stereocilia bundle is wider and the stereocilia are longer relative to OHCs. The nucleus of the IHC is less distinct and sits in a slightly more medial location inside the cell body as compared to the nucleus of the OHC. Deiters cells have a distinct phalangeal process visible as a thin projection from the cell body. The cell body is rounded or lemon-shaped and larger than the cell bodies of IHCs or OHCs. The nucleus is easily visible. Cell classification is corroborated at the transcript level by comparing the abundance of differentially expressed transcripts. Shown is the total number of reads calculated by summing expression levels from each of the four cells shown to the left. Slc26a5 (prestin) is strongly expressed in OHCs but not in IHCs or Deiters cells; Slc17a8 has been reported to be the most strongly differentially expressed gene between IHCs and OHCs. We identified Bace2 as strongly and consistently differentially expressed in Deiters cells as compared to hair cells.

An analysis of the gene expression profile of these IHC, OHC and Deiter's cell types shows that many genes on the OtoSCOPE® platform contain novel exons that are expressed in the inner ear. It was also determined that gene transcripts expressed in the inner ear include novel exons to impart to the translated proteins inner ear-specific function.

In certain embodiments, the present invention relates to a new method to identify genetic regions important for comprehensive diagnosis of genetic deafness, particularly novel exonic regions within genes known to cause non-syndromic deafness and syndromic deafness like Usher syndrome, and visual impairment/blindness. Single cell RNA-sequencing results from individually isolated inner hair cells, outer hair cells, and Deiters cells, were aligned using the STAR aligner software. Results for each individual cell were pooled together in silico. Each pooled group of inner hair cells, outer hair cells and Deiters cells was analyzed using the integrated genome viewer (IGV) software and a tool called Sashimi Plot to visualize reads mapping across splice junctions. All genes on the Otoscope V8 panel (Table 2) were analyzed manually and novel exons were identified visually. Novel exons were checked against existing databases including RefSeq and Ensembl. Regions that were not annotated in these databases were classified as novel.

TABLE 2
Genes causally related to autosomal recessive and
autosomal dominant hearing loss
Autosomal Recessive Genes

| Gene | Locus/Type |
|---|---|
| ADCY1 | DFNB44 |
| BDP1 | |
| BSND | DFNB73/BARTS4A |
| CABP2 | DFNB93 |
| CDC14A | DFNB105 |
| CDH23 | DFNB12/USHID |
| CIB2 | DFNB48 |
| CLDN14 | DFNB29 |
| CLIC5 | DFNB103 |
| CLPP | PRLTS3 |
| COL11A2 | DFNB53/ DFNA13/STL3 |
| DCDC2 | DFNB66 |
| ELMOD3 | DFNB88 |
| EPS8 | DFNB102 |
| EPS8L2 | — |
| ESPN | DFNB36 |
| ESRRB | DFNB35 |
| FAM65B | DFNB104 |
| GIPC3 | DFNB15/ DFNB95 |
| GJB2 | DFNB1/ DFNA3 |
| GJB3 | DFNA2B |
| GJB6 | DFNB1B/ DFNA3B |
| GPSM2 | CMCS |
| GRXCRI | DFNB25 |
| GRXCR2 | DFNB101 |
| HGF | DFNB39 |
| ILDRI | DFNB42 |
| KARS | DFNB89 |
| LHFPL5 | DFNB67 |
| LOXHDI | DFNB77 |
| LRTOMT | DFNB63 |
| MARVELD2 | DFNB49 |
| MET | DFNB97 |
| MSRB3 | DFNB74 |
| MYO3A | DFNB30 |
| MYO6 | DFNB37/ DFNA22 |
| MYO7A | DFNB2/ DFNA11/USH1B |
| MYO15A | DFNB3 |
| NARS2 | DFNB94 |
| OTOA | DFNB22 |
| OTOF | DFNB89 |
| OTOG | DFNB18B |
| OTOGL | DFNB84B |
| PCDH15 | DFNB23/USHIF |
| PDZD7 | DFNB57 |
| PJVK | DFNB59 |
| PNPTI | DFNB70 |
| PTPRO | DFNB84A |
| RDX | DFNB24 |
| RORI | — |
| SIPR2 | DFNB68 |
| SERPINB6 | DFNB91 |
| SLC22A4 | DFNB60 |
| SLC26A4 | DFNB4/PDS |
| SLC26A5 | DFNB61 |
| STRC | DFNB16 |
| SYNE4 | DFNB76 |
| TBC1D24 | DFNB86/ DFNA65 |
| TECTA | DFNB21/ DFNA8/DFNA12 |
| TMC1 | DFNB7/ DFNB11/ DFA36 |
| TMEM132E | DFNB99 |
| TMIE | DFNB6 |
| TMPRSS3 | DFNB8/ DFNB10 |
| TPRN | DFNB79 |
| TRIOBP | DFNB28 |
| TSPEAR | DFNB98 |
| USHIC | DFNB18A/USHIC |
| WHRN | DFNB31/USH2D |
| Total | 68 |

Autosomal Dominant Genes

| Gene | Locus/Type |
|---|---|
| ACTG1 | DFNA20/26 |
| CCDC50 | DFNA44 |
| CD164 | DFNA66 |
| CEACAM16 | DFNA4B |
| COCH | DFNA9 |
| COL11AI | DFNA37/STL2 (AD) |
| COL11A2 | DFNA53/DFNA13/STL3 |
| CRYM | DFNA40 |
| DFNA5 | DFNA5 |
| DIABLO | DFNA64 |
| DIAPHI | DFNA1 |
| DSPP | DGI1 |
| EYA4 | DFNA10 |
| GJB2 | DFNA1/ DFNA3 |
| GJB3 | DFNA2B |
| GJB6 | DFNB1B/ DFNA3B |
| GRHL2 | DFNA28 |
| HOMER2 | DFNA68 |
| KCNO4 | DFNA2A |
| MCM2 | DFNA70 |
| MYH14 | DFNA4A |
| MYH9 | DFNA17 |
| MYO6 | DFNB37/DFNA22 |
| MYO7A | DFNB2/ DFNA11/USH1B |
| NLRP3 | MWS |
| OSBPL2 | DFNA67 |
| P2RX2 | DFNA41 |
| POU4F3 | DFNA15 |
| SIX1 | BOR3/DFNA23 |
| SLC17A8 | DFNA25 |
| TBC1D24 | DFNA86/ DFNA65 |
| TECTA | DFNB21/ DFNA8/ DFNA12 |
| TECTB | — |
| TJP2 | DFNA51 |
| TMC1 | DFNB7/ DFNB11/ DFNA36 |
| TNC | DFNA56 |
| WFS1 | DFNA6/ DFNA14/WFS1 |
| Total | 37 |

Additional Genes Affecting Hearing Loss have been Newly Identified:
AIFM1: Auditory neuropathy spectrum disorder (ANSD) is a form of hearing loss in which auditory signal transmission from the inner ear to the auditory nerve and brain stem is distorted, giving rise of speech perception difficulties beyond that expected for the observed degree of hearing loss. By performing whole exome sequencing on two families segregating ANSD, two disease-causing missense mutations in AIFM1 were identified: c. 1352G>A (p.R451Q) and c.1030C>T (p.L344F). A large cohort of ANSD probands was then screened and nine more missense mutations in AIFM1 were identified. This study implicates variants in AIFM1 as a common cause of ANSD and provides insight into the expanded spectrum of AIFM1-associated diseases. In addition, the finding of cochlear nerve hypoplasia in some patients with AIFM1-related ANSD suggests that cochlea implantation in these patients may have limited success.
HOMER2: a single variant, p.R185P, in HOMER2 was identified as the cause of hearing loss in an extended family segregating ADNSHL. The p.R185P amino acid change alters a highly conserved residue in the coiled-coil domain of HOMER2 that is essential for protein multimerization and the HOMER2-CDC42 interaction. As a scaffolding protein, HOMER2 is involved in intracellular calcium homeostasis and cytoskeletal organization. Consistent with this function, robust expression was found in stereocilia of hair cells in the murine inner ear and it was observed that over-expression of mutant p.P185 HOMER2 mRNA causes anatomical changes of the inner ear and neuromasts in zebrafish embryos. It was also observed that mouse mutants homozygous for the targeted deletion of Homer 2 present with early-onset rapidly progressive hearing loss.
TBC1D24: After excluding mutations in all known deafness genes, segregation mapping and whole exome sequencing were used to identify a unique variant, p.S178L, in TBC1D24 as the cause of hearing loss in an extended family segregating ADNSHL. TBC1D24 encodes a GTPase-activating protein expressed in the cochlea. Variants in this gene have been associated with a variety of clinical symptoms including epileptic disorders, DOORS syndrome (deafness associated with onychodystrophy, osteodystrophy, mental retardation and seizures) and autosomal recessive NSHL (ARNSHL).

Comprehensive genetic testing with a panel like the OtoSCOPE® platform is now the best test to order in the evaluation of hearing loss. An underlying genetic cause for hearing loss can be identified in nearly half of all persons. However in many persons in whom a genetic cause for hearing loss is suspected, genetic testing is negative. This result suggests that: 1) there are additional genes awaiting discovery that cause deafness; 2) there are additional exons that were not currently known in the genes currently implicated in deafness. Using the present technique, novel exons in genes on the OtoSCOPE® platform were discovered (see Table 1).

### Novel Genetic Causes of Autosomal Dominant Non-Syndromic Hearing Loss (ADNSHL)

In certain embodiments, the present invention provides a list of genetic regions important for comprehensive diagnosis of genetic deafness. These regions are novel exonic regions within genes known to cause both non-syndromic deafness and syndromic deafness like Usher syndrome. This list of genetic regions is novel and these regions have not been previously identified as exonic or meaningful for the diagnosis of deafness. These regions are useful for the use in clinical diagnostic testing for genetic hearing loss, Usher syndrome, and visual impairment/blindness.

The exons of the genes included on the OtoSCOPE® platform were identified most commonly by querying expression libraries from diverse tissues such as brain, liver, lung, heart and kidney. These data have shown that the majority of protein-coding genes in vertebrates have multiple alternatively spliced mRNA transcripts that give rise to translated proteins with tissue-specific function. The organ of Corti in the inner ear is the receptor apparatus for hearing. This masterpiece of microarchitecture is made up of a large number of highly specialized types of cells including two kinds of hair cells (inner hair cells (IHCs), outer hair cells (OHCs)), five types of supporting cells (Hensen's cells, Deiters' cells, pillar cells, inner phalangeal cells, border cells), three types of cells in the stria vascularis (marginal, intermediate and basal cells), two types of cells in Reissner's membrane (endolymphatic- and perilymphatic-exposed) and four types of fibrocytes (type I-IV), as well as other cells, the coordinated function of which is to make hearing possible. It is therefore expected that gene transcripts expressed in the inner ear may include novel exons to impart to the translated proteins inner ear-specific function.

Novel genetic causes of hearing loss have been identified, and are presented in Table 1.

TABLE 1
Novel new exons = 18
Total # of Genes = 12

| Mouse | Conserved | In | | |
|---|---|---|---|---|
| Gene | in Humans? | Ensembl? | Mouse (mm10) Location | Human (hg19) Location |
| Cabp2 | Yes | no | chr19: 4, 081, 450-4, 081, 607 | chr11: 67, 292, 851-67293045 |
| Eps8 | Yes | no | chr6: 137, 530, 542-137, 530, 592 | chr12: 15, 825, 388-15, 825, 438 |
| Eps8 | yes | no | chr6: 137, 498, 345-137, 498, 380 | chr12: 15, 792, 360-15, 792, 395 |
| Eps8 | Yes | no | chr6: 137, 493, 548-137, 493, 571 | chr12: 15, 787, 673-15, 787, 696 |
| Myo7a | Yes | no | chr7: 98, 111, 310-98, 111, 522 | chr11: 76, 849, 258-76, 849, 463 |
| Myo7a | Yes | no | chr7: 98, 090, 360-98, 090, 413 | chr11: 76877917-76878106 |
| Otof | Yes | no | chr5: 30, 407, 915-30, 408, 063 | chr2: 26, 727, 461-26, 727, 733 |
| Triobp | Yes | no | chr15: 78, 957, 305-78, 957, 465 | chr22: 38, 105, 438-38105593 |
| Ush1c | Yes | no | chr7: 46, 197, 846-46, 197, 940 | chr11: 17, 519, 066-17, 519, 165 |
| Coch | yes | no | chr12: 51, 594, 118-51, 594, 298 | chr14: 31, 344, 766-31344969 |
| Myh14 | Yes | Yes (mice), | chr7: 44, 669, 243-44, 669, 392 | chr19: 50, 708, 659-50, 708, 811 |
| | | No (human) | | |
| Tectb | Yes | Yes (mice), | chr19: 55, 180, 726-55, 180, 816 | chr10: 114, 043, 164-114, 043, 253 |
| | | No (human) | | |
| Smpx | Yes | Yes (mice), | chrX: 157, 702, 696-157, 702, 737 | chrX: 21, 772, 641-21, 772, 685 |
| | | No (human) | | |
| Smpx | Yes | No | chrX: 157, 702, 769-157, 702, 848 | chrX: 21, 772, 530-21, 772, 609 |
| Eya1 | Yes | No | chr1: 14, 306, 518-14, 306, 791 | chr8: 72, 270, 765-72, 271, 038 |
| Cacna1d | Yes | No | chr14: 30, 085, 906-30, 086, 570 | chr3: 53, 799, 479-53, 800, 143 |
| Cacna1d | Yes | No | chr14: 30, 094, 161-30, 094, 206 | chr3: 53, 788, 928-53, 788, 971 |
| Cacna1d | Yes | No | chr14: 30, 107, 653-30, 107, 712 | chr3: 53, 774, 446-53, 774, 505 |

### Treatment of Genetic Hearing Loss

In certain embodiments, the present invention is broadly applicable to the treatment of genetic hearing loss (also called “hereditary hearing loss”). In certain embodiments, the following method is used:

First, an artificial micro RNA (miRNA) is used to suppress expression of both alleles of the gene carrying the deafness-causing genetic mutation(s). Suppression is based on the concept of RNA interference. In the case of autosomal recessive non-syndromic hearing loss (ARNSHL), both alleles of the gene of interest carrying deafness-causing mutations, and the miRNA reduces expression of both. In the case of autosomal dominant non-syndromic hearing loss (ADNSHL), one allele of the gene of interest carries a deafness-causing mutation while the other allele does not (it is a normal allele). The miRNA reduces expression of both the mutation-carrying allele and the normal allele.

Second, introduction of an exogenous engineered wild-type allele to provide a source of the requisite protein. The engineered allele contains synonymous nucleotide changes that do NOT change the protein sequence but do render the allele resistant to RNA-interference mediated suppression by the artificial miRNA introduced to suppress both alleles of the endogenous gene. The artificial miRNA and engineered allele could be delivered simultaneously in a single vector or separately, in each of two vectors.

In certain embodiments, the present invention provides a method of treating a subject with genetic hearing loss by administering to the subject a nucleic acid, an expression cassette, a vector, or a composition as described herein so as to treat the genetic hearing loss.

The present invention provides in certain embodiments a method of treating genetic hearing loss (GHL) in a patient in need thereof comprising: (a) identifying a mutation in an GHL-causing gene, wherein the mutation causes GHL in the patient; (b) preparing a GHL therapeutic miRNA, wherein the GHL therapeutic miRNA is of 18 to 25 nucleotides in length and knocks-down the GHL-causing gene function at a higher level than it knocks-down gene function in a corresponding wild-type gene; (c) administering to the patient a pharmaceutical composition comprising the GHL therapeutic miRNA and a pharmaceutically acceptable carrier.

In the clinical setting, to identify the specific GHL gene and mutation, a comprehensive genetic panel of genes that causes non-syndromic deafness is screened using targeted genomic enrichment with massively parallel sequencing. Relevant to the heterogeneity of GHL, nearly 75% of identified mutations will be novel—that is to say, a genetic change will be identified that has not been previously reported or described in the scientific literature.

Once a specific mutation has been identified, multiple miRNAs are made, each of which incorporates the identified mutation at a slightly different position in the miRNA structure. These miRNAs are tested to identify the specific miRNA that most potently knocks-down the ADNSHL-causing gene function while having minimal knock-down effect on the corresponding wild-type gene.

In certain embodiments, the GHL-causing gene is an exon listed in Table 1.

The present invention provides in certain embodiments a method of treating genetic hearing loss (GHL) in a patient in need thereof comprising administering to a patient identified as having a mutation in one of the exons listed in Table 1 a pharmaceutical composition comprising pharmaceutically acceptable carrier and a GHL therapeutic miRNA, wherein the miRNA is of 18 to 25 nucleotides in length and knocks-down the mutant gene function at a higher level than it knocks-down gene function in a corresponding wild-type gene.

In certain embodiments, the miRNA is of 20 to 22 nucleotides in length. In certain embodiments, the miRNA is 21 nucleotides in length.

In certain embodiments, the miRNA knocks-down the GHL-causing gene function by at least 50% more than it knocks-down the corresponding wild-type gene function.

In certain embodiments, the pharmaceutical composition further comprises an shRNA or siRNA.

In certain embodiments, the miRNA is contained in an expression cassette comprising a promoter operably linked to a nucleic acid encoding the miRNA. In certain embodiments, the promoter is a polII or polIII promoter (such as an H1 or U6 promoter). In certain embodiments, the promoter is a tissue-specific promoter. In certain embodiments, the promoter is an inducible promoter.

In certain embodiments, the expression cassette further comprises a marker gene (such as green fluorescent protein (GFP)).

In certain embodiments, the expression cassette is contained in a vector.

In certain embodiments, the vector is an adeno-associated virus (AAV) vector, an adenovirus vector or a bovine AAV vector.

In certain embodiments, the pharmaceutical composition is administered intravenously and/or directly into the patient's inner ear.

The present invention provides a method of suppressing the accumulation of gene product from an GHL-causing gene in a cell by introducing nucleic acid molecules (e.g., a ribonucleic acid (RNA)) described herein into the cell in an amount sufficient to suppress accumulation of the GHL-causing gene product in the cell. In certain embodiments, the accumulation of gene product is suppressed by at least 10%. In certain embodiments, the accumulation of gene product is suppressed by at least 10%, 20%, 30%, 40%, 50%, 60%, 70%, 80%, 90% 95%, or 99%. In certain embodiments, the suppression of the accumulation of the protein is in an amount sufficient to cause a therapeutic effect, e.g., to reduce the GHL.

The present invention provides a method to inhibit expression of an GHL-causing gene in a cell by introducing a nucleic acid molecule (e.g., a ribonucleic acid (RNA)) described herein into the cell in an amount sufficient to inhibit expression of the GHL-causing gene product, and wherein the RNA inhibits expression of the GHL-causing gene. In certain embodiments, the GHL-causing gene product is inhibited by at least 10%, 20%, 30%, 40%, 50%, 60%, 70%, 80%, 90% 95%, or 99%.

The present invention provides a viral vector comprising a promoter and a micro RNA (miRNA) specific for a target sequence. In certain embodiments, the promoter is an inducible promoter. In certain embodiments, the vector is an adenoviral, lentiviral, adeno-associated viral (AAV), poliovirus, HSV, or murine Maloney-based viral vector. In certain embodiments, the targeted sequence is a sequence associated with ADNSHL.

The present invention also provides a method to inhibit expression of a protein associated with GHL in a mammal in need thereof, by introducing the vector encoding a miRNA described herein into a cell in an amount sufficient to inhibit expression of the GHL-causing gene product. The GHL-causing gene product is inhibited by at least 10%, 20%, 30%, 40%, 50%, 60%, 70%, 80%, 90% 95%, or 99%.

This invention relates to compounds, compositions, and methods useful for modulating GHL gene expression using miRNA molecules. This invention also relates to compounds, compositions, and methods useful for modulating the expression and activity of other genes involved in pathways of GHL gene expression and/or activity by RNA interference (RNAi) using small nucleic acid molecules. An “RNA interference” or “RNAi” molecule; “small interfering RNA,” “short interfering RNA” or “siRNA” molecule; “short hairpin RNA” or “shRNA” molecule; or “miRNA” molecule is a RNA duplex of nucleotides that is targeted to a nucleic acid sequence of interest. An “RNA duplex” refers to the structure formed by the complementary pairing between two regions of a RNA molecule. An RNAi molecule is “targeted” to a gene in that the nucleotide sequence of the duplex portion of the RNAi molecule is complementary to a nucleotide sequence of the targeted gene. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length. In particular, the instant invention features small nucleic acid molecules, such as short interfering nucleic acid (siNA), short interfering RNA (siRNA), double-stranded RNA (dsRNA), micro-RNA (miRNA), and short hairpin RNA (shRNA) molecules and methods used to modulate the expression of GHL genes. An RNA molecule of the instant invention can be, e.g., chemically synthesized, expressed from a vector or enzymatically synthesized.

As used herein when a claim indicates an RNA “corresponding to” it is meant the RNA that has the same sequence as the DNA, except that uracil is substituted for thymine.

The present invention further provides a method of substantially silencing a target gene of interest or targeted allele for the gene of interest in order to provide a therapeutic effect. As used herein the term “substantially silencing” or “substantially silenced” refers to decreasing, reducing, or inhibiting the expression of the target gene or target allele by at least about 5%, 10%, 15%, 20%, 25%, 30%, 35%, 40%, 45%, 50%, 55%, 60%, 65%, 70%, 75%, 80%, 85% to 100%. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the condition. Therapeutic effects can be measured quantitatively by a physician or qualitatively by a patient afflicted with the hearing loss targeted by the miRNA. In certain embodiments wherein both the mutant and wild-type allele are substantially silenced, the term therapeutic effect defines a condition in which silencing of the wild-type allele's expression does not have a deleterious or harmful effect on normal functions such that the patient would not have a therapeutic effect.

In one embodiment, the invention features a method for treating or preventing GHL in a subject or organism comprising contacting the subject or organism with an miRNA of the invention under conditions suitable to modulate the expression of the GHL gene in the subject or organism whereby the treatment or prevention of GHL can be achieved. The miRNA molecule of the invention can be expressed from vectors as described herein or otherwise known in the art to target appropriate tissues or cells in the subject or organism.

In one embodiment, the invention features a method for treating or preventing GHL in a subject or organism comprising, contacting the subject or organism with an miRNA molecule of the invention via local administration to relevant tissues or cells, for example, by administration of vectors or expression cassettes of the invention that provide miRNA molecules of the invention to relevant cells.

Methods of delivery of viral vectors include, but are not limited to, intravenous administration and administration directly into a patient's inner ear. Generally, AAV virions may be introduced into cells using either in vivo or in vitro transduction techniques. If transduced in vitro, the desired recipient cell will be removed from the subject, transduced with AAV virions and reintroduced into the subject. Alternatively, syngeneic or xenogeneic cells can be used where those cells will not generate an inappropriate immune response in the subject.

In one embodiment, pharmaceutical compositions will comprise sufficient genetic material to produce a therapeutically effective amount of the miRNA of interest, i.e., an amount sufficient to reduce or ameliorate symptoms of the disease state in question or an amount sufficient to confer the desired benefit. The pharmaceutical compositions may also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

As is apparent to those skilled in the art in view of the teachings of this specification, an effective amount of viral vector which must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

It should be understood that more than one transgene could be expressed by the delivered viral vector. Alternatively, separate vectors, each expressing one or more different transgenes, can also be delivered as described herein. Furthermore, it is also intended that the viral vectors delivered by the methods of the present invention be combined with other suitable compositions and therapies.

The present invention further provides miRNA or shRNA, an expression cassette and/or a vector as described herein for use in medical treatment or diagnosis.

The present invention provides the use of an miRNA or shRNA, an expression cassette and/or a vector as described herein to prepare a medicament useful for treating GHL.

The present invention also provides a nucleic acid, expression cassette, vector, or composition of the invention for use in therapy.

The present invention also provides a nucleic acid, expression cassette, vector, or composition of the invention for treating GHL.

“Treating” as used herein refers to ameliorating at least one symptom of, curing and/or preventing the development of hearing loss. In certain embodiment of the invention, RNAi molecules are employed to inhibit expression of a target gene. By “inhibit expression” is meant to reduce, diminish or suppress expression of a target gene. Expression of a target gene may be inhibited via “gene silencing.” Gene silencing refers to the suppression of gene expression, e.g., transgene, heterologous gene and/or endogenous gene expression, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner via RNA interference, thereby preventing translation of the gene's product.

Disclosed herein is a strategy that results in substantial silencing of targeted genes via Rnai. Use of this strategy results in markedly diminished in vitro and in vivo expression of targeted genes. This strategy is useful in reducing expression of targeted genes in order to provide therapy for GHL. As used herein the term “substantial silencing” means that the mRNA of the targeted gene is inhibited and/or degraded by the presence of the introduced miRNA, such that expression of the targeted gene is reduced by about 10% to 100% as compared to the level of expression seen when the miRNA is not present. Generally, when an gene is substantially silenced, it will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction expression as compared to when the miRNA is not present. As used herein the term “substantially normal activity” means the level of expression of a gene when an miRNA has not been introduced to a cell.

### RNA Interference (RNAi) Molecules

RNAi directs sequence-specific gene silencing by double-stranded RNA (dsRNA) which is processed into functional small inhibitory RNAs (˜21nt). In nature, RNAi for regulation of gene expression occurs primarily via small RNAs known as microRNAs (miRNAs). Mature microRNAs (˜19-25 nts) are processed from larger primary miRNA transcripts (pri-miRNAs) which contain stem-loop regions. Via a series of processing events catalyzed by the ribonucleases, Drosha and Dicer, the miRNA duplex region is liberated and a single strand (the antisense “guide” strand) is then incorporated into the RNA Induced Silencing Complex (RISC), thus generating a functional complex capable of base-pairing with and silencing target transcripts. The mode of target repression primarily depends upon the degree of complementarity; transcript cleavage typically requires a high-degree of base-pairing, whereas translational repression and mRNA destabilization occurs when small RNAs bind imperfectly to target transcripts (most often in the 3′ UTR). Indeed for the latter, short stretches of complementarity—as little as 6 bp—may be sufficient to cause gene silencing.

An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, an GHIL-causing gene. An “RNA duplex” refers to the structure formed by the complementary pairing between two regions of a RNA molecule. As used herein the term “miRNA” encompasses both the naturally occurring miRNA sequences as well as artificially generated miRNA shuttle vectors.

The miRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

“Knock-down,” “knock-down technology” refers to a technique of gene silencing in which the expression of a target gene is reduced as compared to the gene expression prior to the introduction of the miRNA, which can lead to the inhibition of production of the target gene product. The term “reduced” is used herein to indicate that the target gene expression is lowered by 1-100%. In other words, the amount of RNA available for translation into a polypeptide or protein is minimized. For example, the amount of protein may be reduced by 10, 20, 30, 40, 50, 60, 70, 80, 90, 95, or 99%. In some embodiments, the expression is reduced by about 90% (i.e., only about 10% of the amount of protein is observed a cell as compared to a cell where miRNA molecules have not been administered).

According to a method of the present invention, the expression of an GHL-causing gene product can be modified via RNA interference. For example, the accumulation of a gene product can be suppressed in a cell. The term “suppressing” refers to the diminution, reduction or elimination in the number or amount of transcripts present in a particular cell. For example, the accumulation of mRNA encoding GHL-causing gene product can be suppressed in a cell by RNA interference (RNAi).

A mutant protein refers to the protein encoded by a gene having a mutation, e.g., a missense or nonsense mutation in the targeted GHL-causing gene product. A mutant GHL-causing gene may be disease-causing, i.e., may lead to a disease associated with the presence of GHL-causing gene product in an animal having either one or two mutant allele(s).

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome.

The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

A “nucleotide sequence” is a polymer of DNA or RNA that can be single-stranded or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The invention encompasses isolated or substantially purified nucleic acid nucleic acid molecules and compositions containing those molecules. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences are also encompassed by the present invention. By “fragment” or “portion” is meant a full length or less than full length of the nucleotide sequence.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

A “variant” or “mutant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

A “transgene” refers to a gene that has been introduced into the genome by transformation. Transgenes include, for example, DNA that is either heterologous or homologous to the DNA of a particular cell to be transformed. Additionally, transgenes may include native genes inserted into a non-native organism, or chimeric genes. The term “endogenous gene” refers to a native gene in its natural location in the genome of an organism.

“Wild-type” refers to the normal gene or organism found in nature.

“Genome” refers to the complete genetic material of an organism.

A “vector” is defined to include, inter alia, any viral vector, as well as any plasmid, cosmid, phage or binary vector in double or single stranded linear or circular form that may or may not be self transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. The coding region usually codes for a functional RNA of interest, for example an miRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions. An “intron” is a sequence of RNA that is contained in the primary transcript but is removed through cleavage and re-ligation of the RNA within the cell to create the mature mRNA that can be translated into a protein.

“Functional RNA” refers to sense RNA, antisense RNA, ribozyme RNA, miRNA, or other RNA that may not be translated but yet has an effect on at least one cellular process.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell.

“cDNA” refers to a single- or a double-stranded DNA that is complementary to and derived from mRNA.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted herein, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Examples of promoters that may be used in the present invention include the mouse U6 RNA promoters, synthetic human H1RNA promoters, SV40, CMV, RSV, RNA polymerase II and RNA polymerase III promoters.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of miRNA constructs, expression may refer to the transcription of the miRNA only. In addition, expression refers to the transcription and stable accumulation of sense (mRNA) or functional RNA. Expression may also refer to the production of protein.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

“Antisense inhibition” refers to the production of antisense RNA transcripts capable of suppressing the expression of protein from an endogenous gene or a transgene.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

“Chromosomally-integrated” refers to the integration of a foreign gene or nucleic acid construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells.

“Transformed,” “transduced,” “transgenic” and “recombinant” refer to a host cell into which a heterologous nucleic acid molecule has been introduced. As used herein the term “transfection” refers to the delivery of DNA into eukaryotic (e.g., mammalian) cells. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

The miRNAs of the present invention can be generated by any method known to the art, for example, by in vitro transcription, recombinantly, or by synthetic means. In one example, the miRNAs can be generated in vitro by using a recombinant enzyme, such as T7 RNA polymerase, and DNA oligonucleotide templates.

### MicroRNA Shuttles for RNAi

Artificial miRNA shuttle vectors are used to mimic natural miRNAs and suppress the mutated ADNSHL gene of interest. miRNA shuttles closely recapitulate natural miRNA structures, are predictably processed and are amenable to control by tissue-specific and/or regulated promoters. They are outstanding for long-term RNA interference studies to prevent progression of ADNHSL by suppressing expression of the mutated gene. miRNAs are small cellular RNAs (˜22nt) that are processed from precursor stem loop transcripts. Known miRNA stem loops can be modified to contain RNAi sequences specific for genes of interest. miRNA molecules can be preferable over shRNA molecules because miRNAs are endogenously expressed. Therefore, miRNA molecules are unlikely to induce dsRNA-responsive interferon pathways, they are processed more efficiently than shRNAs, and they have been shown to silence 80% more effectively.

### Nucleic Acid Molecules of the Invention

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. The RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

### Expression Cassettes of the Invention

The present invention also provides an expression cassette comprising a sequence encoding miRNA molecule.

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

In certain embodiments, the expression cassette further contains a promoter. In certain embodiments, the promoter is a regulatable promoter. In certain embodiments, the promoter is a constitutive promoter. In certain embodiments, the promoter is a PGK, CMV or RSV promoter.

The present invention provides a vector containing the expression cassette described above. In certain embodiments, the vector is a viral vector. In certain embodiments, the viral vector is an adenoviral, lentiviral, adeno-associated viral (AAV), poliovirus, HSV, or murine Maloney-based viral vector.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

A “chimeric” vector or expression cassette, as used herein, means a vector or cassette including nucleic acid sequences from at least two different species, or has a nucleic acid sequence from the same species that is linked or associated in a manner that does not occur in the “native” or wild-type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the miRNA, or the like. Such elements may be included in the DNA as desired to obtain the optimal performance of the miRNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein.

The recombinant DNA can be readily introduced into the host cells, e.g., mammalian, bacterial, yeast or insect cells by transfection with an expression vector composed of DNA encoding the miRNA by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described herein below, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like. See, for example, U.S. Pat. Nos. 5,350,674 and 5,585,362.

As discussed herein, a “transfected” “or “transduced” host cell or cell line is one in which the genome has been altered or augmented by the presence of at least one heterologous or recombinant nucleic acid sequence. The host cells of the present invention are typically produced by transfection with a DNA sequence in a plasmid expression vector, a viral expression vector, or as an isolated linear DNA sequence. The transfected DNA can become a chromosomally integrated recombinant DNA sequence, which is composed of sequence encoding the miRNA.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the preselected DNA segment is being expressed. Expression may be evaluated by specifically identifying the peptide products of the introduced recombinant DNA sequences or evaluating the phenotypic changes brought about by the expression of the introduced recombinant DNA segment in the host cell.

The instant invention provides a cell expression system for expressing exogenous nucleic acid material in a mammalian recipient. The expression system, also referred to as a “genetically modified cell,” comprises a cell and an expression vector for expressing the exogenous nucleic acid material. The genetically modified cells are suitable for administration to a mammalian recipient, where they replace the endogenous cells of the recipient. Thus, the preferred genetically modified cells are non-immortalized and are non-tumorigenic.

According to one embodiment, the cells are transfected or otherwise genetically modified ex vivo. The cells are isolated from a mammal (preferably a human), nucleic acid introduced (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

As used herein, “exogenous nucleic acid material” refers to a nucleic acid or an oligonucleotide, either natural or synthetic, which is not naturally found in the cells; or if it is naturally found in the cells, is modified from its original or native form. Thus, “exogenous nucleic acid material” includes, for example, a non-naturally occurring nucleic acid that can be transcribed into an anti-sense RNA, a miRNA, as well as a “heterologous gene” (i.e., a gene encoding a protein that is not expressed or is expressed at biologically insignificant levels in a naturally-occurring cell of the same type). To illustrate, a synthetic or natural gene encoding human erythropoietin (EPO) would be considered “exogenous nucleic acid material” with respect to human peritoneal mesothelial cells since the latter cells do not naturally express EPO. Still another example of “exogenous nucleic acid material” is the introduction of only part of a gene to create a recombinant gene, such as combining an regulatable promoter with an endogenous coding sequence via homologous recombination.

The condition amenable to gene inhibition therapy may be a prophylactic process, i.e., a process for preventing disease or an undesired medical condition. Thus, the instant invention embraces a system for delivering miRNA that has a prophylactic function (i.e., a prophylactic agent) to the mammalian recipient.

Methods for Introducing the Expression Cassettes of the Invention into Cells

The inhibitory nucleic acid material (e.g., an expression cassette encoding miRNA directed to a gene of interest) can be introduced into the cell ex vivo or in vivo by genetic transfer methods, such as transfection or transduction, to provide a genetically modified cell. Various expression vectors (i.e., vehicles for facilitating delivery of exogenous nucleic acid into a target cell) are known to one of ordinary skill in the art.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including calcium phosphate DNA co-precipitation, DEAE-dextran, electroporation, cationic liposome-mediated transfection, tungsten particle-facilitated microparticle bombardment, and strontium phosphate DNA co-precipitation.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

The exogenous nucleic acid material can include the nucleic acid encoding the miRNA together with a promoter to control transcription. The promoter characteristically has a specific nucleotide sequence necessary to initiate transcription. The exogenous nucleic acid material may further include additional sequences (i.e., enhancers) required to obtain the desired gene transcription activity. For the purpose of this discussion an “enhancer” is simply any non-translated DNA sequence that works with the coding sequence (in cis) to change the basal transcription level dictated by the promoter. The exogenous nucleic acid material may be introduced into the cell genome immediately downstream from the promoter so that the promoter and coding sequence are operatively linked so as to permit transcription of the coding sequence. An expression vector can include an exogenous promoter element to control transcription of the inserted exogenous gene. Such exogenous promoters include both constitutive and regulatable promoters.

Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the beta-actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain an regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

Accordingly, the amount of miRNA generated in situ is regulated by controlling such factors as the nature of the promoter used to direct transcription of the nucleic acid sequence, (i.e., whether the promoter is constitutive or regulatable, strong or weak) and the number of copies of the exogenous nucleic acid sequence encoding a miRNA sequence that are in the cell.

In addition to at least one promoter and at least one heterologous nucleic acid sequence encoding the miRNA, the expression vector may include a selection gene, for example, a neomycin resistance gene, for facilitating selection of cells that have been transfected or transduced with the expression vector.

Cells can also be transfected with two or more expression vectors, at least one vector containing the nucleic acid sequence(s) encoding the miRNA(s), the other vector containing a selection gene. The selection of a suitable promoter, enhancer, selection gene, and/or signal sequence is deemed to be within the scope of one of ordinary skill in the art without undue experimentation.

The following discussion is directed to various utilities of the instant invention. For example, the instant invention has utility as an expression system suitable for silencing the expression of gene(s) of interest.

The instant invention also provides methods for genetically modifying cells of a mammalian recipient in vivo. According to one embodiment, the method comprises introducing an expression vector for expressing an miRNA sequence in cells of the mammalian recipient in situ by, for example, injecting the vector into the recipient.

### Delivery Vehicles for the Expression Cassettes of the Invention

Delivery of compounds into tissues can be limited by the size and biochemical properties of the compounds. Currently, efficient delivery of compounds into cells in vivo can be achieved only when the molecules are small (usually less than 600 Daltons).

The selection and optimization of a particular expression vector for expressing a specific miRNA in a cell can be accomplished by obtaining the nucleic acid sequence of the miRNA, possibly with one or more appropriate control regions (e.g., promoter, insertion sequence); preparing a vector construct comprising the vector into which is inserted the nucleic acid sequence encoding the miRNA; transfecting or transducing cultured cells in vitro with the vector construct; and determining whether the miRNA is present in the cultured cells.

Vectors for cell gene therapy include viruses, such as replication-deficient viruses (described in detail below). Exemplary viral vectors are derived from Harvey Sarcoma virus, ROUS Sarcoma virus, (MPSV), Moloney murine leukemia virus and DNA viruses (e.g., adenovirus).

Replication-deficient retroviruses are capable of directing synthesis of all virion proteins, but are incapable of making infectious particles. Accordingly, these genetically altered retroviral expression vectors have general utility for high-efficiency transduction of nucleic acid sequences in cultured cells, and specific utility for use in the method of the present invention. Such retroviruses further have utility for the efficient transduction of nucleic acid sequences into cells in vivo. Retroviruses have been used extensively for transferring nucleic acid material into cells. Protocols for producing replication-deficient retroviruses (including the steps of incorporation of exogenous nucleic acid material into a plasmid, transfection of a packaging cell line with plasmid, production of recombinant retroviruses by the packaging cell line, collection of viral particles from tissue culture media, and infection of the target cells with the viral particles) are well known in the art.

An advantage of using retroviruses for gene therapy is that the viruses insert the nucleic acid sequence encoding the miRNA into the host cell genome, thereby permitting the nucleic acid sequence encoding the miRNA to be passed on to the progeny of the cell when it divides. Promoter sequences in the LTR region have can enhance expression of an inserted coding sequence in a variety of cell types. Some disadvantages of using a retrovirus expression vector are (1) insertional mutagenesis, i.e., the insertion of the nucleic acid sequence encoding the miRNA into an undesirable position in the target cell genome which, for example, leads to unregulated cell growth and (2) the need for target cell proliferation in order for the nucleic acid sequence encoding the miRNA carried by the vector to be integrated into the target genome.

Another viral candidate useful as an expression vector for transformation of cells is the adenovirus, a double-stranded DNA virus. The adenovirus is infective in a wide range of cell types, including, for example, muscle and endothelial cells.

Adenoviruses (Ad) are double-stranded linear DNA viruses with a 36 kb genome. Several features of adenovirus have made them useful as transgene delivery vehicles for therapeutic applications, such as facilitating in vivo gene delivery. Recombinant adenovirus vectors have been shown to be capable of efficient in situ gene transfer to parenchymal cells of various organs, including the lung, brain, pancreas, gallbladder, and liver. This has allowed the use of these vectors in methods for treating inherited genetic diseases, such as cystic fibrosis, where vectors may be delivered to a target organ.

Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Several approaches traditionally have been used to generate the recombinant adenoviruses. One approach involves direct ligation of restriction endonuclease fragments containing a nucleic acid sequence of interest to portions of the adenoviral genome. Alternatively, the nucleic acid sequence of interest may be inserted into a defective adenovirus by homologous recombination results. The desired recombinants are identified by screening individual plaques generated in a lawn of complementation cells.

Application of miRNA is generally accomplished by transfection of synthetic miRNAs, in vitro synthesized RNAs, or plasmids expressing miRNAs. More recently, viruses have been employed for in vitro studies and to generate transgenic mouse knock-downs of targeted genes. Recombinant adenovirus, adeno-associated virus (AAV) and feline immunodeficiency virus (FIV) can be used to deliver genes in vitro and in vivo. Each has its own advantages and disadvantages. Adenoviruses are double stranded DNA viruses with large genomes (36 kb) and have been engineered by my laboratory and others to accommodate expression cassettes in distinct regions.

Adeno-associated viruses have encapsidated genomes, similar to Ad, but are smaller in size and packaging capacity (˜30 nm vs. ˜100 nm; packaging limit of ˜4.5 kb). AAV contain single stranded DNA genomes of the + or the − strand. Eight serotypes of AAV (1-8) have been studied extensively. An important consideration for the present application is that AAV5 transduces striatal and cortical neurons, and is not associated with any known pathologies.

Adeno associated virus (AAV) is a small nonpathogenic virus of the parvoviridae family. AAV is distinct from the other members of this family by its dependence upon a helper virus for replication. In the absence of a helper virus, AAV may integrate in a locus specific manner into the q arm of chromosome 19. The approximately 5 kb genome of AAV consists of one segment of single stranded DNA of either plus or minus polarity. The ends of the genome are short inverted terminal repeats which can fold into hairpin structures and serve as the origin of viral DNA replication. Physically, the parvovirus virion is non-enveloped and its icosohedral capsid is approximately 20 nm in diameter.

Further provided by this invention are chimeric viruses where AAV can be combined with herpes virus, herpes virus amplicons, baculovirus or other viruses to achieve a desired tropism associated with another virus. For example, the AAV4 ITRs could be inserted in the herpes virus and cells could be infected. Post-infection, the ITRs of AAV4 could be acted on by AAV4 rep provided in the system or in a separate vehicle to rescue AAV4 from the genome. Therefore, the cellular tropism of the herpes simplex virus can be combined with AAV4 rep mediated targeted integration. Other viruses that could be utilized to construct chimeric viruses include lentivirus, retrovirus, pseudotyped retroviral vectors, and adenoviral vectors.

Also provided by this invention are variant AAV vectors. For example, the sequence of a native AAV, can be modified at individual nucleotides. The present invention includes native and mutant AAV vectors. The present invention further includes all AAV serotypes.

FIV is an enveloped virus with a strong safety profile in humans; individuals bitten or scratched by FIV-infected cats do not seroconvert and have not been reported to show any signs of disease. Like AAV, FIV provides lasting transgene expression in mouse and nonhuman primate neurons, and transduction can be directed to different cell types by pseudotyping, the process of exchanging the virus's native envelope for an envelope from another virus.

Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene silencing therapy and the optimization of the conditions for insertion of the selected expression vector into the cell, are within the scope of one of ordinary skill in the art without the need for undue experimentation.

In another embodiment, the expression vector is in the form of a plasmid, which is transferred into the target cells by one of a variety of methods: physical (e.g., microinjection, electroporation, scrape loading, microparticle bombardment) or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (Promega®, Madison, Wis.). However, the efficiency of transfection by these methods is highly dependent on the nature of the target cell and accordingly, the conditions for optimal transfection of nucleic acids into cells using the herein-mentioned procedures must be optimized. Such optimization is within the scope of one of ordinary skill in the art without the need for undue experimentation.

### Dosages, Formulations and Routes of Administration of the Agents of the Invention

The agents of the invention are preferably administered so as to result in a reduction in at least one symptom associated with a disease. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems, which are well known to the art. As used herein, the term “therapeutic miRNA” refers to any miRNA that has a beneficial effect on the recipient. Thus, “therapeutic miRNA” embraces both therapeutic and prophylactic miRNA.

Administration of miRNA may be accomplished through the administration of the nucleic acid molecule encoding the miRNA. Pharmaceutical formulations, dosages and routes of administration for nucleic acids are generally known.

The present invention envisions treating ADNSHL in a mammal by the administration of an agent, e.g., a nucleic acid composition, an expression vector, or a viral particle of the invention. Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the inner ear. Alternatively the therapeutic agent may be introduced systemically (e.g., intraveneously). In another example, the therapeutic agent may be introduced intramuscularly for viruses that traffic back to affected neurons from muscle, such as AAV, lentivirus and adenovirus. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules, as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0, saline solutions and water.

### Adeno Associated Virus (AAV)

Adeno associated virus (AAV) is a small nonpathogenic virus of the parvoviridae family. AAV is distinct from the other members of this family by its dependence upon a helper virus for replication. The approximately 5 kb genome of AAV consists of one segment of single stranded DNA of either plus or minus polarity. The ends of the genome are short inverted terminal repeats which can fold into hairpin structures and serve as the origin of viral DNA replication. Physically, the parvovirus virion is non-enveloped and its icosohedral capsid is approximately 20 nm in diameter.

To date, numerous serologically distinct AAVs have been identified, and more than a dozen have been isolated from humans or primates. The genome of AAV2 is 4680 nucleotides in length and contains two open reading frames (ORFs). The left ORF encodes the non-structural Rep proteins, Rep 40, Rep 52, Rep 68 and Rep 78, which are involved in regulation of replication and transcription in addition to the production of single-stranded progeny genomes. Furthermore, two of the Rep proteins have been associated with the preferential integration of AAV genomes into a region of the q arm of human chromosome 19. Rep68/78 has also been shown to possess NTP binding activity as well as DNA and RNA helicase activities. The Rep proteins possess a nuclear localization signal as well as several potential phosphorylation sites. Mutation of one of these kinase sites resulted in a loss of replication activity.

The ends of the genome are short inverted terminal repeats (ITR) which have the potential to fold into T-shaped hairpin structures that serve as the origin of viral DNA replication. Within the ITR region two elements have been described which are central to the function of the ITR, a GAGC repeat motif and the terminal resolution site (trs). The repeat motif has been shown to bind Rep when the ITR is in either a linear or hairpin conformation. This binding serves to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The AAV virion is a non-enveloped, icosohedral particle approximately 25 nm in diameter, consisting of three related proteins referred to as VP1, VP2 and VP3. The right ORF encodes the capsid proteins VP1, VP2, and VP3. These proteins are found in a ratio of 1:1:10 respectively and are all derived from the right-hand ORF. The capsid proteins differ from each other by the use of alternative splicing and an unusual start codon. Deletion analysis has shown that removal or alteration of VP1 which is translated from an alternatively spliced message results in a reduced yield of infections particles. Mutations within the VP3 coding region result in the failure to produce any single-stranded progeny DNA or infectious particles. An AAV particle is a viral particle comprising an AAV capsid protein. An AAV capsid polypeptide can encode the entire VP1, VP2 and VP3 polypeptide. The particle can be a particle comprising AAV2 and other AAV capsid proteins (i.e., a chimeric protein, such as AAV2 and AAV9).

An AAV2 particle is a viral particle comprising an AAV2 capsid protein. An AAV2 capsid polypeptide encoding the entire VP1, VP2, and VP3 polypeptide can overall have at least about 63% homology (or identity) to the polypeptide having the amino acid sequence encoded by nucleotides set forth in NC_001401 (nucleotide sequence encoding AAV2 capsid protein). The capsid protein can have about 70% homology, about 75% homology, 80% homology, 85% homology, 90% homology, 95% homology, 98% homology, 99% homology, or even 100% homology to the protein encoded by the nucleotide sequence set forth in NC_001401. The capsid protein can have about 70% identity, about 75% identity, 80% identity, 85% identity, 90% identity, 95% identity, 98% identity, 99% identity, or even 100% identity to the protein encoded by the nucleotide sequence set forth in NC_001401. The particle can be a particle comprising another AAV and AAV2 capsid protein, i.e., a chimeric protein. Variations in the amino acid sequence of the AAV2 capsid protein are contemplated herein, as long as the resulting viral particle comprising the AAV2 capsid remains antigenically or immunologically distinct from AAV4, as can be routinely determined by standard methods. Specifically, for example, ELISA and Western blots can be used to determine whether a viral particle is antigenically or immunologically distinct from AAV1. Furthermore, the AAV2 viral particle preferably retains tissue tropism distinction from AAV1, such as that exemplified in the examples herein, though an AAV2 chimeric particle comprising at least one AAV2 coat protein may have a different tissue tropism from that of an AAV2 particle consisting only of AAV2 coat proteins.

In certain embodiments, the invention further provides an AAV2 particle containing, i.e., encapsidating, a vector comprising a pair of AAV2 inverted terminal repeats. The nucleotide sequence of AAV2 ITRs is known in the art. Furthermore, the particle can be a particle comprising both AAV1 and AAV2 capsid protein, i.e., a chimeric protein. Moreover, the particle can be a particle encapsidating a vector comprising a pair of AAV inverted terminal repeats from other AAVs (e.g., AAV1-AAV9 and AAVrh10). The vector encapsidated in the particle can further comprise an exogenous nucleic acid inserted between the inverted terminal repeats.

The following features of AAV have made it an attractive vector for gene transfer. AAV vectors have been shown in vitro to stably integrate into the cellular genome; possess a broad host range; transduce both dividing and non-dividing cells in vitro and in vivo and maintain high levels of expression of the transduced genes. Viral particles are heat stable, resistant to solvents, detergents, changes in pH, temperature, and can be concentrated on CsCl gradients or by other means. The present invention provides methods of administering AAV particles, recombinant AAV vectors, and recombinant AAV virions. For example, an AAV2 particle is a viral particle comprising an AAV2 capsid protein, or an AAV9 particle is a viral particle comprising an AAV9 capsid protein. A recombinant AAV2 vector is a nucleic acid construct that comprises at least one unique nucleic acid of AAV2. A recombinant AAV2 virion is a particle containing a recombinant AAV2 vector. To be considered within the term “AAV2 ITRs” the nucleotide sequence must retain one or both features described herein that distinguish the AAV2 ITR from the AAV1 ITR: (1) three (rather than four as in AAV1) “GAGC” repeats and (2) in the AAV2 ITR Rep binding site the fourth nucleotide in the first two “GAGC” repeats is a C rather than a T.

The promoter to drive expression of the protein or the sequence encoding another agent to be delivered can be any desired promoter, selected by known considerations, such as the level of expression of a nucleic acid functionally linked to the promoter and the cell type in which the vector is to be used. Promoters can be an exogenous or an endogenous promoter. Promoters can include, for example, known strong promoters such as SV40 or the inducible metallothionein promoter, or an AAV promoter, such as an AAV p5 promoter. Additional examples of promoters include promoters derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), etc. Additional examples include regulated promoters.

The AAV vector can further comprise an exogenous (heterologous) nucleic acid functionally linked to the promoter. By “heterologous nucleic acid” is meant that any heterologous or exogenous nucleic acid can be inserted into the vector for transfer into a cell, tissue or organism. The nucleic acid can encode a polypeptide or protein or an antisense RNA, for example. By “functionally linked” is meant such that the promoter can promote expression of the heterologous nucleic acid, as is known in the art, such as appropriate orientation of the promoter relative to the heterologous nucleic acid. Furthermore, the heterologous nucleic acid preferably has all appropriate sequences for expression of the nucleic acid, as known in the art, to functionally encode, i.e., allow the nucleic acid to be expressed. The nucleic acid can include, for example, expression control sequences, such as an enhancer, and necessary information processing sites, such as ribosome binding sites, RNA splice sites, polyadenylation sites, and transcriptional terminator sequences. The nucleic acid can encode more than one gene product, limited only by the size of nucleic acid that can be packaged.

In certain embodiments of the present invention, the heterologous nucleic acid can encode beneficial proteins that replace missing or defective proteins required by the subject into which the vector in transferred, such as Rheb or Rhes.

The term “polypeptide” as used herein refers to a polymer of amino acids and includes full-length proteins and fragments thereof. Thus, “protein” and “polypeptide” are often used interchangeably herein. Substitutions can be selected by known parameters to be neutral. As will be appreciated by those skilled in the art, the invention also includes those polypeptides having slight variations in amino acid sequences or other properties. Such variations may arise naturally as allelic variations (e.g. due to genetic polymorphism) or may be produced by human intervention (e.g., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules. These modifications can result in changes in the amino acid sequence, provide silent mutations, modify a restriction site, or provide other specific mutations.

The present method provides a method of delivering a nucleic acid to a cell comprising administering to the cell an AAV particle containing a vector comprising the nucleic acid inserted between a pair of AAV inverted terminal repeats, thereby delivering the nucleic acid to the cell. Administration to the cell can be accomplished by any means, including simply contacting the particle, optionally contained in a desired liquid such as tissue culture medium, or a buffered saline solution, with the cells. The particle can be allowed to remain in contact with the cells for any desired length of time, and typically the particle is administered and allowed to remain indefinitely. For such in vitro methods, the virus can be administered to the cell by standard viral transduction methods, as known in the art and as exemplified herein. Titers of virus to administer can vary, particularly depending upon the cell type, but will be typical of that used for AAV transduction in general. Additionally the titers used to transduce the particular cells in the present examples can be utilized. The cells can include any desired cell in humans as well as other large (non-rodent) mammals, such as primates, horse, sheep, goat, pig, and dog.

More specifically, the present invention provides a method of delivering a nucleic acid to a cell in the brain, particularly medium spiny neurons, comprising the nucleic acid inserted between a pair of AAV inverted terminal repeats, thereby delivering the nucleic acid to the cell.

The present invention further provides a method of delivering a nucleic acid to a cell in a subject comprising administering to the subject an AAV particle comprising the nucleic acid inserted between a pair of AAV inverted terminal repeats, thereby delivering the nucleic acid to a cell in the subject.

Also provided is a method of delivering a nucleic acid to a brain cell, such as a neuron in the striatum or cortex in a subject comprising administering to the subject an AAV particle comprising the nucleic acid inserted between a pair of AAV inverted terminal repeats, thereby delivering the nucleic acid to the neuron or other cell in the subject.

Certain embodiments of the present disclosure provide a cell comprising a viral vector as described herein.

### AAV Vectors

In one embodiment, a viral vector of the disclosure is an AAV vector. An “AAV” vector refers to an adeno-associated virus, and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are eight known serotypes of primate AAVs, AAV-1 to AAV-9 and AAVrh10. For example, serotype AAV2 is used to refer to an AAV which contains capsid proteins encoded from the cap gene of AAV2 and a genome containing 5′ and 3′ ITR sequences from the same AAV2 serotype. As used herein, for example, rAAV1 may be used to refer an AAV having both capsid proteins and 5′-3′ ITRs from the same serotype or it may refer to an AAV having capsid proteins from one serotype and 5′-3′ ITRs from a different AAV serotype, e.g., capsid from AAV serotype 2 and ITRs from AAV serotype 5. For each example illustrated herein the description of the vector design and production describes the serotype of the capsid and 5′-3′ ITR sequences. The abbreviation “rAAV” refers to recombinant adeno-associated virus, also referred to as a recombinant AAV vector (or “rAAV vector”).

An “AAV virus” or “AAV viral particle” refers to a viral particle composed of at least one AAV capsid protein (preferably by all of the capsid proteins of a wild-type AAV) and an encapsidated polynucleotide. If the particle comprises heterologous polynucleotide (i.e., a polynucleotide other than a wild-type AAV genome such as a transgene to be delivered to a mammalian cell), it is typically referred to as “rAAV”.

In one embodiment, the AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional AAV ITR sequences.

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

In one embodiment, AAV ITRs can be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

In one embodiment, AAV capsids can be derived from AAV2. Suitable DNA molecules for use in AAV vectors will be less than about 5 kilobases (kb), less than about 4.5 kb, less than about 4 kb, less than about 3.5 kb, less than about 3 kb, less than about 2.5 kb in size and are known in the art.

In one embodiment, the selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Such promoter sequences are commercially available from, e.g., Stratagene (San Diego, Calif.).

Examples of heterologous promoters include the CMV promoter. Examples of inducible promoters include DNA responsive elements for ecdysone, tetracycline, hypoxia and aufin.

In one embodiment, the AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art.

Alternatively, AAV ITRs can be excised from the viral genome or from an AAV vector containing the same and fused 5′ and 3′ of a selected nucleic acid construct that is present in another vector using standard ligation techniques. For example, ligations can be accomplished in 20 mM Tris-Cl pH 7.5, 10 mM MgCl₂, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs.

Additionally, chimeric genes can be produced synthetically to include AAV ITR sequences arranged 5′ and 3′ of one or more selected nucleic acid sequences. The complete chimeric sequence is assembled from overlapping oligonucleotides prepared by standard methods.

In order to produce rAAV virions, an AAV expression vector is introduced into a suitable host cell using known techniques, such as by transfection. A number of transfection techniques are generally known in the art. See, e.g., Sambrook et al. (1989) Molecular Cloning, a laboratory manual, Cold Spring Harbor Laboratories, New York. Particularly suitable transfection methods include calcium phosphate co-precipitation, direct micro-injection into cultured cells, electroporation, liposome mediated gene transfer, lipid-mediated transduction, and nucleic acid delivery using high-velocity microprojectiles.

In one embodiment, suitable host cells for producing rAAV virions include microorganisms, yeast cells, insect cells, and mammalian cells, that can be, or have been, used as recipients of a heterologous DNA molecule. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) can be used in the practice of the present disclosure. Particularly, the human cell line 293 is a human embryonic kidney cell line that has been transformed with adenovirus type-5 DNA fragments, and expresses the adenoviral E1a and E1b genes. The 293 cell line is readily transfected, and provides a particularly convenient platform in which to produce rAAV virions.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. Suitable homologues of the AAV rep coding region include the human herpesvirus 6 (HHV-6) rep gene which is also known to mediate AAV-2 DNA replication.

By “AAV cap coding region” is meant the art-recognized region of the AAV genome which encodes the capsid proteins VP1, VP2, and VP3, or functional homologues thereof. These Cap expression products supply the packaging functions which are collectively required for packaging the viral genome.

In one embodiment, AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors have been described which encode Rep and/or Cap expression products.

Methods of delivery of viral vectors include injecting the AAV into the subject, such as intravenously by means of the superficial temporal vein.

In one embodiment, pharmaceutical compositions will comprise sufficient genetic material to produce a therapeutically effective amount of the nucleic acid of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water or saline. Additionally, auxiliary substances, such as pH buffering substances, and the like, may be present in such vehicles. A thorough discussion of pharmaceutically acceptable excipients is available in Remington's Pharmaceutical Sciences (Mack Pub. Co., N.J. 1991).

It should be understood that more than one transgene could be expressed by the delivered viral vector. Alternatively, separate vectors, each expressing one or more different transgenes, can also be delivered to the subject as described herein. Furthermore, it is also intended that the viral vectors delivered by the methods of the present disclosure be combined with other suitable compositions and therapies.

As is apparent to those skilled in the art in view of the teachings of this specification, an effective amount of viral vector which must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

In certain embodiments, the rAAV is administered at a dose of about 0.3-2 ml of 1×10⁵-1×10¹⁶vg/ml. In certain embodiments, the rAAV is administered at a dose of about 1-3 ml of 1×10⁷-1×10¹⁴vg/ml. In certain embodiments, the rAAV is administered at a dose of about 1-2 ml of 1×10⁸-1×10¹³vg/ml.

Formulations containing the rAAV particles will contain an effective amount of the rAAV particles in a vehicle, the effective amount being readily determined by one skilled in the art. The rAAV particles may typically range from about 1% to about 95% (w/w) of the composition, or even higher or lower if appropriate. The quantity to be administered depends upon factors such as the age, weight and physical condition of the animal or the human subject considered for treatment. Effective dosages can be established by one of ordinary skill in the art through routine trials establishing dose response curves. The subject is treated by administration of the rAAV particles in one or more doses. Multiple doses may be administered as is required to maintain adequate enzyme activity.

Vehicles including water, aqueous saline, or other known substances can be employed with the subject invention. To prepare a formulation, the purified composition can be isolated, lyophilized and stabilized. The composition may then be adjusted to an appropriate concentration, optionally combined with an anti-inflammatory agent, and packaged for use.

The present invention provides a method of increasing the level of a target protein in a cell by introducing a protein, or nucleic acid molecule encoding a protein described above into a cell in an amount sufficient to increase or decrease the level of the target protein in the cell. In certain embodiments, the accumulation of target protein is increased or decreased by at least 10%. In certain embodiments the reduction of target protein is decreased by at least 10%, 20%, 30%, 40%, 50%, 60%, 70%, 80%, 90% 95%, or 99%. In certain embodiments, the accumulation of target protein is increased by at least 10%, 20%, 30%, 40%, 50%, 60%, 70%, 80%, 90% 95%, or 99%.

### Nucleic Acids Encoding Therapeutic Agents

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of Rheb or Rhes).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

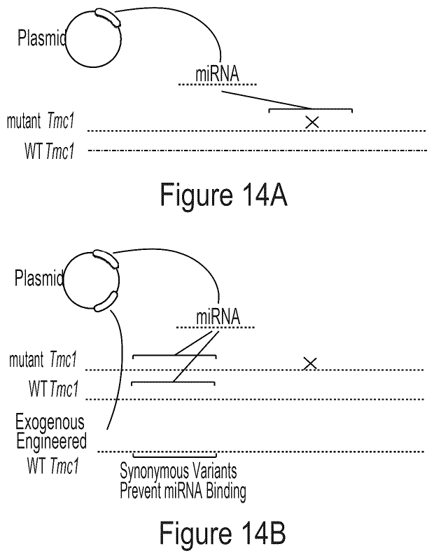

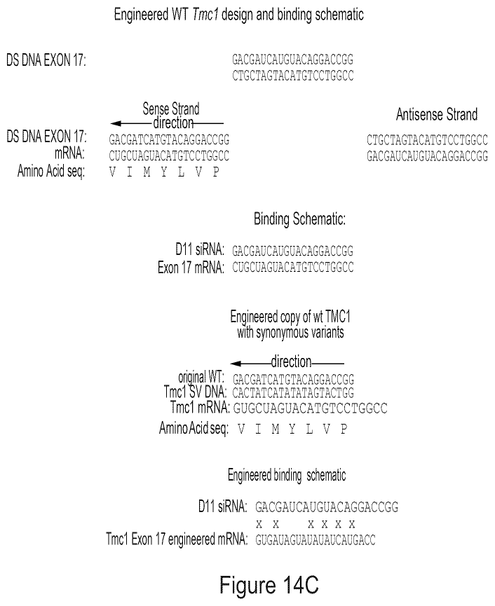

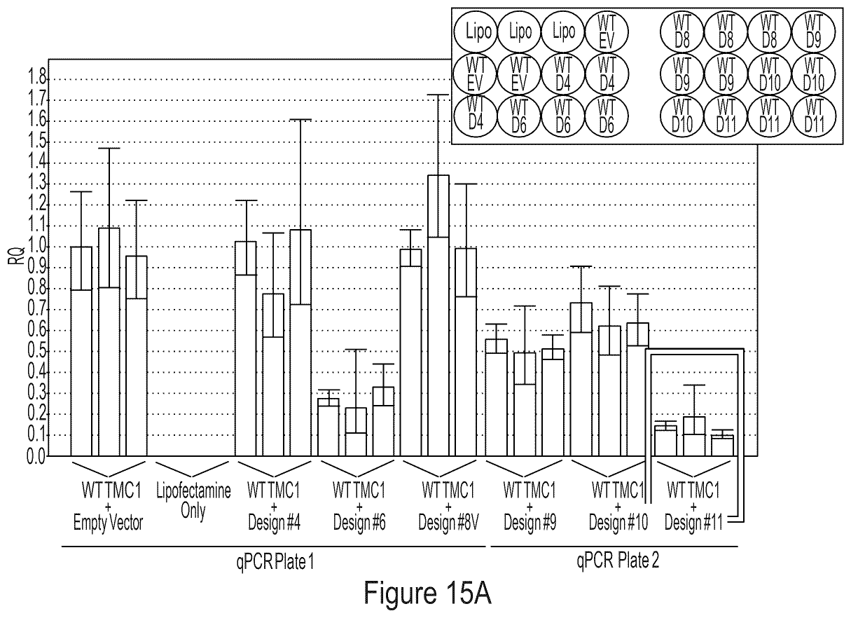

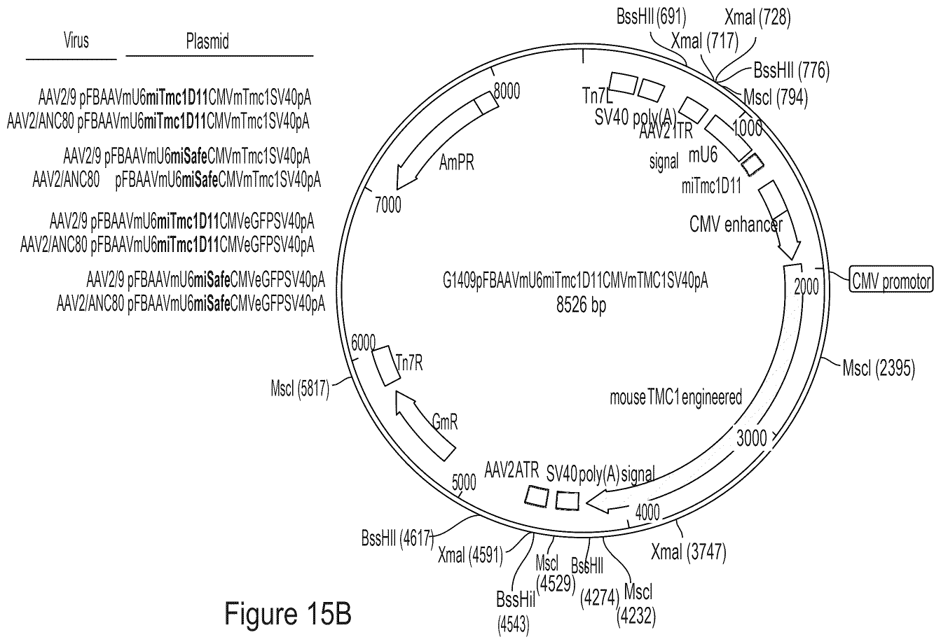

In certain embodiments, the present invention provides a method of treating hearing loss comprising: (a) administering a gene suppression agent that suppresses both copies of an endogenous gene causing the hearing loss; and (b) administering an exogenous wild-type allele engineered to resist suppression by the gene suppression agent. See, FIGS. 14A-14C and 15A-15B.

In certain embodiments, the gene suppression agent is an RNAi molecule.

In certain embodiments, the gene suppression agent is an miRNA.

In certain embodiments, the gene suppression agent is a CRISPR system.

In certain embodiments, the gene suppression agent and the exogenous wild-type allele are administered simultaneously in a single vector.

In certain embodiments, the gene suppression agent and the exogenous wild-type allele are administered separately in a two vectors.

In certain embodiments, the endogenous gene causing the hearing loss is an exon listed in Table 1, Table 2, or is ACTG1, CCDC50, CEACAM1, COCH, COL11A2, CRYM, DFNA5, DIABLO, DIAPH1, DSPP, EYA4, GJB2, GJB3, GJB6, GRHL2, HOMER2, KCNQ4, MYH14, MYH9, MYO1A, MYO6, P2RX, POU4F3, SLC1748, TBC1D24, TECTA, TJP2, TMC1, TNC, or WFS1.

In certain embodiments, the present invention provides a method of treating genetic hearing loss (GHL) in a patient in need thereof comprising administering to a patient identified as having a mutation in a GHL-causing gene a pharmaceutical composition comprising pharmaceutically acceptable carrier and a GHL therapeutic miRNA, wherein the miRNA is of 18 to 25 nucleotides in length and suppresses expression of the GHL-causing gene to a greater level than it suppresses expression of a corresponding wild-type gene, wherein the GHL-causing gene is an exon listed in Table 1.

In certain embodiments, the present invention provides a method of treating a genetic hearing loss (GHL) in a patient in need thereof comprising: (a) identifying a mutation in a GHL-causing gene, wherein the mutation causes GHL in the patient, and wherein the GHL-causing gene is an exon listed in Table 1; and (b) administering to the patient a pharmaceutical composition comprising a therapeutic miRNA and a pharmaceutically acceptable carrier, wherein the GHL therapeutic miRNA is of 18 to 25 nucleotides in length and knocks-down the GHL-causing gene function at a higher level than it knocks-down gene function in a corresponding wild-type gene.

In certain embodiments, the miRNA is of 20 to 22 nucleotides in length.

In certain embodiments, the miRNA is 21 nucleotides in length.

In certain embodiments, the miRNA knocks-down the GHL-causing gene function by at least 50% more than it knocks-down the corresponding wild-type gene function.

In certain embodiments, the pharmaceutical composition further comprises an shRNA or siRNA.

In certain embodiments, the miRNA is contained in an expression cassette comprising a promoter operably linked to a nucleic acid encoding the miRNA.

In certain embodiments, the promoter is a polII or polIII promoter.

In certain embodiments, the promoter is an H1 or U6 promoter.

In certain embodiments, the promoter is a tissue-specific promoter.

In certain embodiments, the promoter is an inducible promoter.

In certain embodiments, the expression cassette further comprises a marker gene.

In certain embodiments, the marker gene is green fluorescent protein (GFP).

In certain embodiments, the expression cassette is contained in a vector.

In certain embodiments, the vector is an adeno-associated virus (AAV) vector, adenovirus vector or bovine AAV vector.

In certain embodiments, the pharmaceutical composition is administered intravenously and/or directly into the patient's inner ear.

In certain embodiments, the present invention provides a method of transducing cochlear epithelial tissue in an animal, comprising administering rAAV comprising a therapeutic agent to the animal, wherein the administration is intravenously and the rAAV crosses the blood-labyrinthine barrier in the animal, and wherein the rAAV2/9 transfects spiral ganglion neurons, inner hair cells, outer hair cells, stria vascularis, and/or vestibular organs.

In certain embodiments, the e therapeutic agent is an RNAi molecule.

In certain embodiments, the RNAi molecule is an miRNA.

In certain embodiments, the rAAV is rAAV2/9.

In certain embodiments, the administration is intravenously by means of superficial temporal vein in the animal.

In certain embodiments, the present invention provides a method of treating hearing loss in a patient in need thereof, comprising administering a viral vector through a round window membrane of the patient, wherein the patient previously received a canalostomy.

In certain embodiments, the present invention provides a method of transducing cochlear tissue in an animal, comprising: (a) making a post-auricular incision, (b) making a hole with an otologic drill in the chchlea bulla and the posterior semicircular canal, (c) puncturing the RWM, and (d) microinjecting a therapeutic agent into a scala tympani.

In certain embodiments, the therapeutic agent is an AAV or Anc80.

In certain embodiments, the AAV is AAV2/9.

In certain embodiments, the present invention provides a method of transducing cochlear tissue in an animal, comprising administering rAAV2/9 intravenously by means of superficial temporal vein to the animal.

In certain embodiments, the present invention provides a method for detecting that a subject is has a gene associated with genetic hearing loss comprising: (a) providing a biological sample from the subject; and (b) contacting the biological sample with at least one first oligonucleotide probe at least 8 nucleotides in length that is complementary to a sequence that comprises an exon listed in Table 1.

The invention will now be illustrated by the following non-limiting Examples.

### Example 1

### Autosomal Dominant Non-Syndromic Hearing Loss

In the evaluation of persons with hearing loss, comprehensive genetic testing is now recognized as the most informative clinical test. Because it offers the highest diagnostic rate, healthcare providers are able to make evidence-based decisions and provide better and more cost-effective patient care. In a recent study of 1119 sequentially accrued patients who presented with sensorineural hearing loss (SNHL) in at least one ear (there were no exclusionary criteria based on age, age of onset, phenotype or previous testing), comprehensive genetic testing using OtoSCOPE® genetic testing panel an underlying genetic cause for hearing loss was identified in 440 patients (39%). Phenotypic diversity impacted the overall diagnostic rate. In patients with symmetric hearing loss and a positive family history, diagnostic rates were 67% and 55% for ADNSHL and autosomal recessive non-syndromic hearing loss (ARNSHL), respectively. Inheritance pattern also impacted the type of causal variant that was identified. If the hearing loss was autosomal dominant, missense variants were diagnosed 85% of the time. In contrast, with autosomal recessive inheritance that figure dropped to 46%. The remaining 54% of variants associated with the diagnosis of ARNSHL included copy number variations (CNVs), indels, nonsense variants and splice variants at 20%, 19%, 9% and 6%, respectively; these latter genetic variants all predict null alleles.

To improve the diagnostic rate for comprehensive genetic testing, targeted efforts would logically focus on the identification of novel exons associated with ear-specific isoforms of all genes currently implicated in hearing loss. However the identification of new exons in these genes is challenging. The cochlea's membranous labyrinth is a masterpiece of delicate microarchitecture comprised of many different and highly specialized cell types. The list includes two types of hair cells (inner hair cells, IHCs; outer hair cells, OHCs), five types of supporting cells (Hensen's cells, Deiters cells, pillar cells, inner phalangeal cells, border cells), three types of strial cells (marginal, intermediate and basal cells), two types of cells in Reissner's membrane (endolymphatic- and perilymphatic-exposed) and four types of fibrocytes (type I-IV), in addition to other cell types, all of which are encased in a bony labyrinth. The latter hampers access, especially after ossification is complete, while the diversity of cell types markedly reduces analytical sensitivity when the membranous labyrinth is studied en masse.

To meet these challenges, a single cell isolation procedure based on a modification of a technique has been established and validated. The bony labyrinth is dissected, the membranous labyrinth is extracted, and then collagenase and mechanical disruption is used to create a single-cell suspension. Individual cells in this suspension are identified, photographed, isolated, washed and collected for single-cell studies.

As discussed in Example 3 below, a ‘gene-targeting’ therapeutic strategy was developed that is applicable to all types of ADNSHL and ARNSHL caused by a given gene. This approach reduces the number of necessary therapeutics from over 7000 (at the mutation-specific level) to approximately 150 (at the gene-specific level), which makes RNAi-based personalized precision medicine for hearing loss practical.

OtoSCOPE®: The OtoSCOPE® gene panel uses targeted-genomic enrichment technology coupled with massively parallel sequencing (TGE+MPS) to provide comprehensive genetic testing for hearing loss. OtoSCOPE® gene panel version 8 captures all exons and splice sites of all genes implicated in NSHL, as well as mitochondrial mutations, several common forms of syndromic hearing loss, and ‘non-syndromic mimics’ like the Usher syndrome genes. Further ‘hidden exon identification’ is used to improve comprehensive genetic testing as a clinical tool in the evaluation of hearing loss.

Single-cell RNA-Seq: Recent advances in ultra-low input RNA-sequencing (RNA-Seq) technologies have enabled transcriptome profiling and pathway analysis of single cells without the need for fluorescence-activated cell sorting (FACS). A rapid and affordable single cell RNA-Seq protocol has been developed and validated to isolate and characterize the transcriptomes of different cell types of the membranous labyrinth, including IHCs, OHCs and supporting cells. The data robustly differentiate between these cell types and demonstrate the expected transcriptional variability between single cells.

RNA Interference and Gene Therapy: It has been shown that a single intra-cochlear injection of an artificial miRNA carried in a viral vector can slow progression of hearing loss for over 35 weeks in the Beethoven mouse, a mouse model of ADNSHL caused by a dominant gain-of-function mutation in the Tmc1 (transmembrane channel-like 1) gene (see US Patent Publication No. 2016-0090597, which is incorporated by reference herein). This study was the first to demonstrate the feasibility of using RNAi to suppress an endogenous deafness-causing allele as a method of preventing ADNSHL.

Transducing Cells in the Adult Mouse Cochlea: In order to determine whether there is a temporal ‘window of opportunity’ during which ADNSHL may be reversible, an operative approach must be developed to permit reliable transduction of the membranous labyrinth in the adult mouse. To date, all successful gene therapy experiments targeting hearing loss in mice have treated animals at P0-2, prior to the onset of hearing. Studies at later time points have not been possible because of technical difficulties in transducing cells in the adult murine cochlea. To address this challenge, a reliable approach has been tested and developed that makes it possible to transduce hair cells in mice of any age.

Data from the Encyclopedia of DNA Elements (ENCODE) project have shown that most of the human genome is transcribed as a complex repertoire of ˜20,000 protein-coding genes, ˜16,000 long non-coding RNAs, ˜10,000 small non-coding RNA and 14,000 pseudogenes. Although the number of protein-coding genes seems low given the proteomic complexity evident in many tissues, the number of isoforms is estimated to be at least 5-10-fold higher. Isoform-specific expression is particularly common in neurologic tissues like the brain and inner ear. As an example of the former, long-read sequence analysis of neurexin expression in adult mouse prefrontal cortex indicates that the three Nrxn genes produce thousands of isoform variants.

MYO15A serves as an example of the untapped complexity of the inner ear transcriptome. Detailed studies by Rehman and colleagues in 2016 identified a novel alternative transcription start site and alternative splicing of cassette exons 8 and 26 (Rehman AU, Bird JE, Faridi R, Shahzad M, Shah S, Lee K, Khan SN, Imtiaz A, Ahmed ZM, Riazuddin S, Santos-Cortez RL, Ahmad W, Leal SM, Riazuddin S, Friedman TB. Mutational spectrum of MYO15A and the molecular mechanisms of DFNB3 human deafness. Hum Mutat 2016 October;37 (10): 991-1003. doi: 10.1002/humu.23042. PMID: 27375115.). Exon 8 is a mini-exon of six conserved nucleotides (ATAAAG) that encode isoleucine and lysine, which become inserted into a surface-exposed flexible region of the motor domain close to the ATP nucleotide binding pocket; cassette exon 26 is transcribed as 18 in-frame codons essential for inner ear function as evidenced by a homozygous mutant allele, p. Trp1975* that co-segregates with deafness in two consanguineous Iranian families. When an upstream alternative acceptor site of exon 26 is utilized, an additional 162 nucleotides are included, which result in premature translation stop codons (PTCs) that truncate the protein after the IQ motifs.

Identifying the transcriptional complexity of MYO15A was labor intensive and challenging, a reflection of both the complexity of the cochlea and the paucity of available tissue to study, especially at the cell-specific level. The challenge of single cell analysis has most commonly been addressed by fluorescence-activated cell sorting (FACS), a technique that uses light scattering to separate individual cells by specific fluorescence signatures. As an alternative approach, a rapid and simple single cell RNA-Seq protocol has been developed to isolate and characterize different cell types in the cochlea.

The Single-Cell Dissection Technique offers several advantages over FACS. First, cell requirement is lower. The 3,500 IHCs and 12,000 OHCs in a single mouse cochlea are vastly outnumbered by the sum total of other cell types making their isolation a challenge. In a recent single-cell RNA-Seq paper by Burns and colleagues, for example, these researchers used FACS to identify and isolate individual hair cells from P1 mouse cochlea (Burns JC, Kelly MC, Hoa M, Morell RJ, Kelley MW. Single-cell RNA-seq resolves cellular complexity in sensory organs from the neonatal inner ear. Nat Commun 2015 October;6:8557. doi: 10.1038/ncomms9557. PMID: 26469390). They isolated 10 hair cells, which they were unable to subcategorize into IHC and OHC groups. In comparison, the present single-cell dissection technique isolated and identified >200 OHCs, >200 IHCs and >200 Deiters cells across multiple time points including P15, P30, P60 and P228.

Second, the Single-Cell Dissection Technique allows for the isolation of hair cells across different time points. Hair cell dissociation is easier early in development (P0-P2) and becomes increasingly more difficult at later time points. Later time points, however, are of great interest. Not only is the onset of hearing in mice at ˜P15, but the effects of many genetic mutations and environmental insults cannot be studied in immature ears.

Speed of isolation is a third advantage. Cells are isolated within 30 minutes of the death of the animal (a self-imposed time limit) and the sample is chilled during first 10 minutes of the cell isolation procedure. Speed and low temperatures minimize changes to the transcriptome and slow transcript degradation. Individual cells are then collected, photographed, and placed in individual tubes. Imaging each cell is standard as it allows cross referencing of cell morphology with the transcriptome (FIG. 1). This type of validation has improved the ability to recognize cells and provides assurance that cell-type assignments are correct. Many FACS experiments, in comparison, pool cells after sorting and then sequence or evaluate cells in groups.

Finally, the Single-Cell Dissection Technique does not depend on fluorescence, which frees investigators of constraints that require either mouse models expressing fluorescent tags or antibodies against unique cell surface markers.

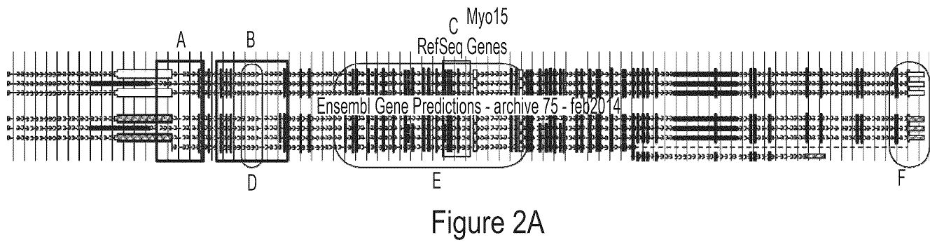

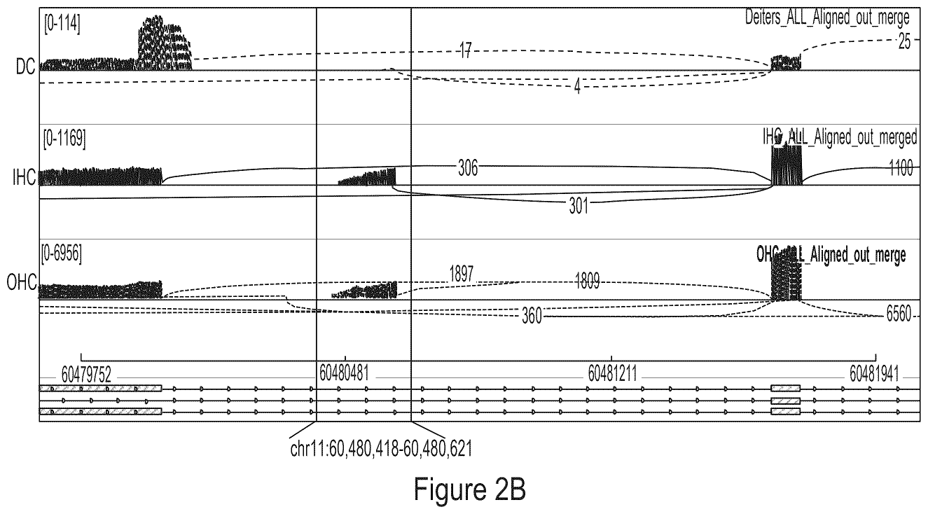

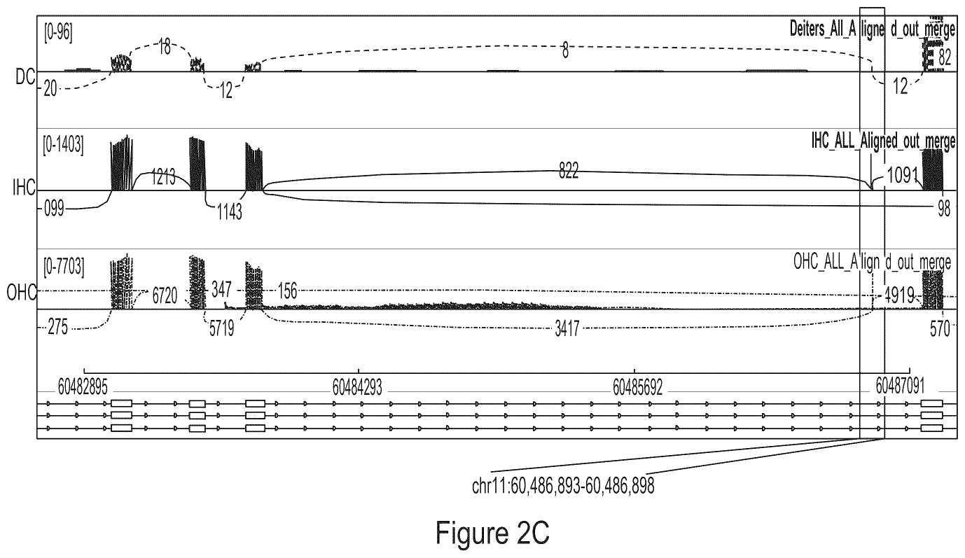

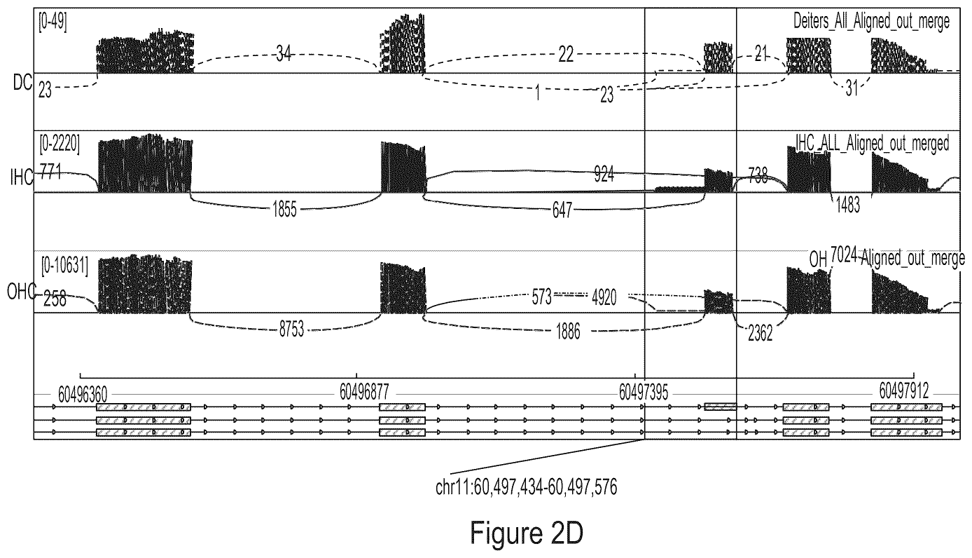

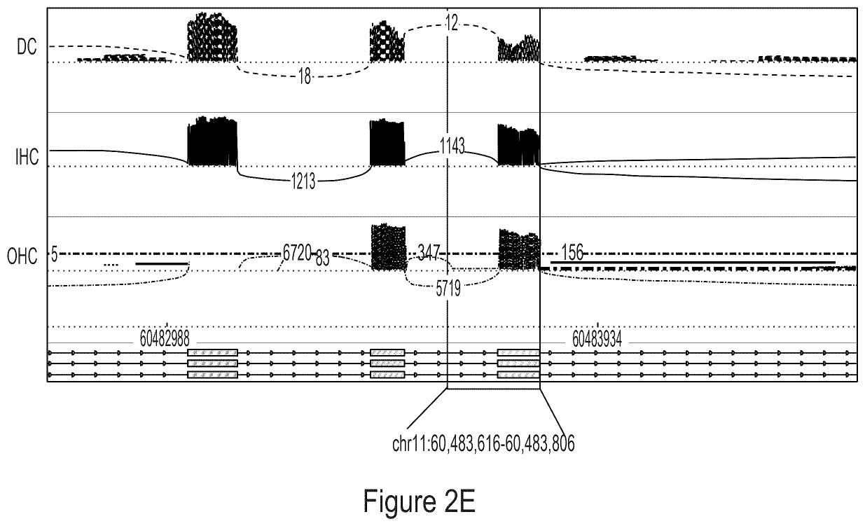

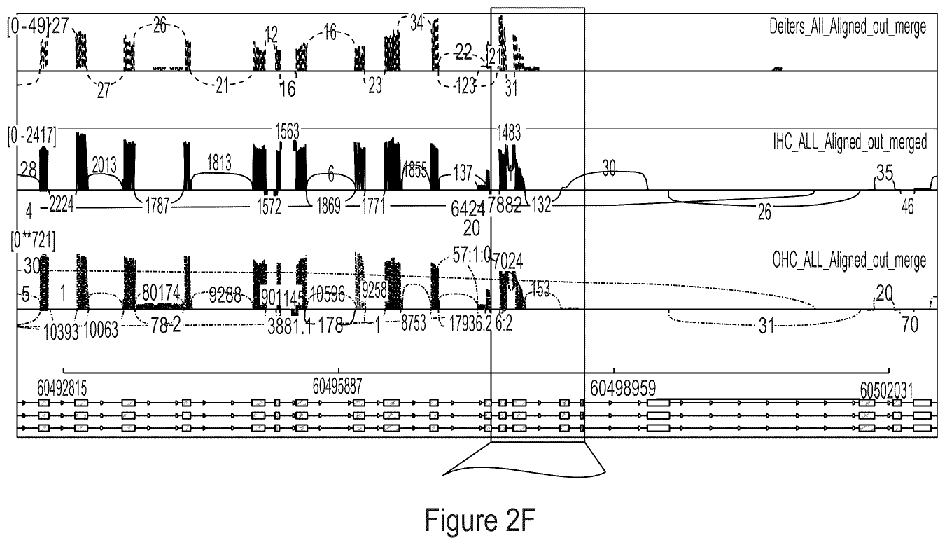

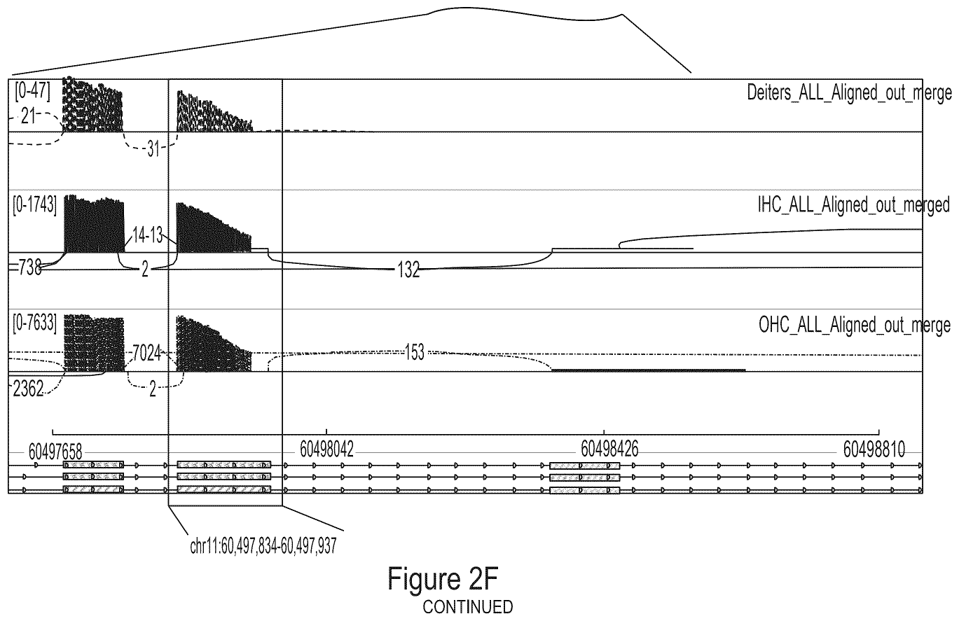

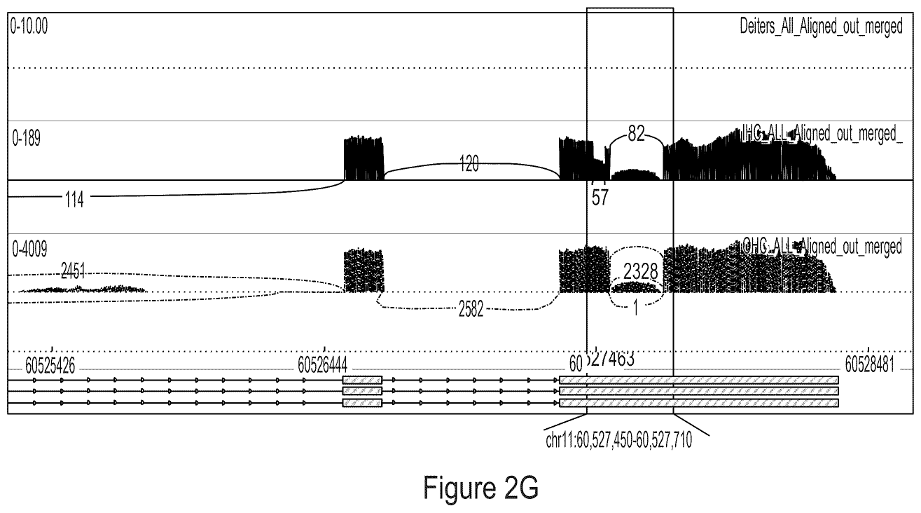

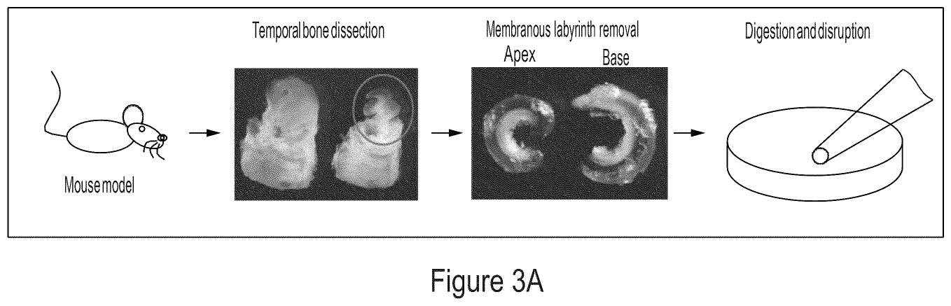

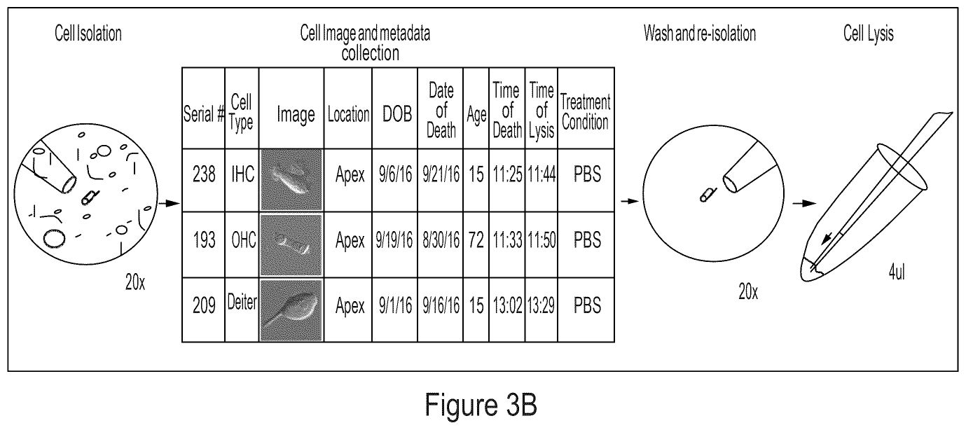

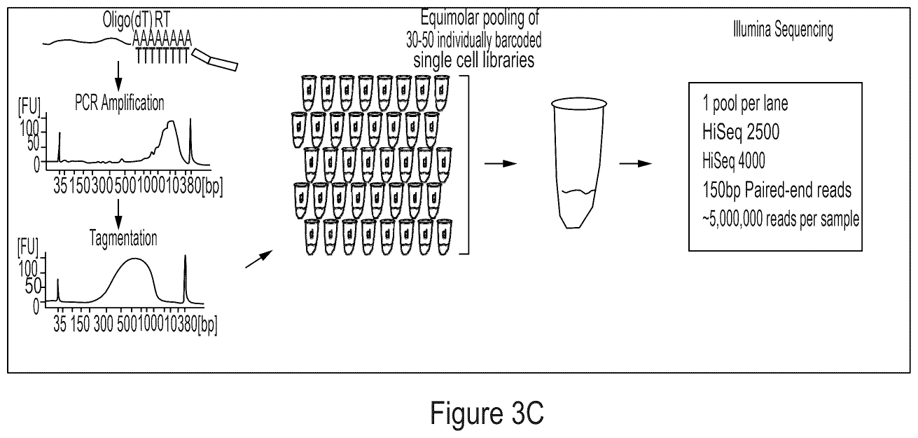

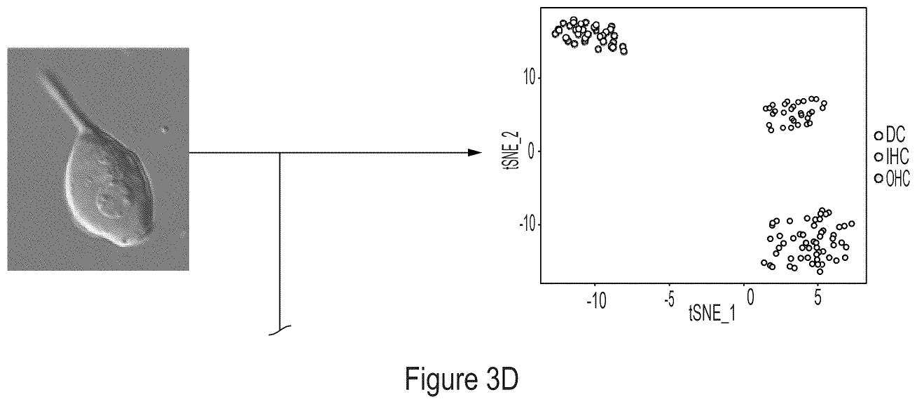

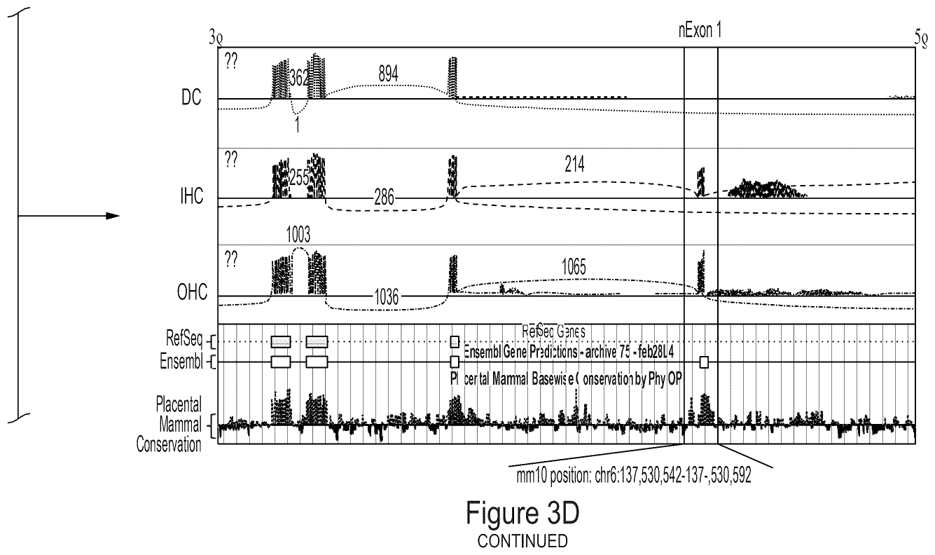

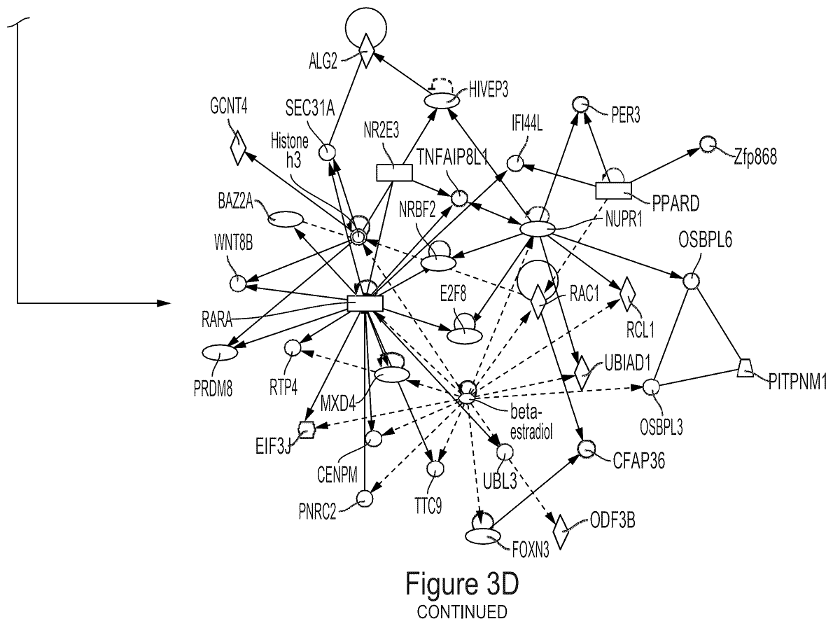

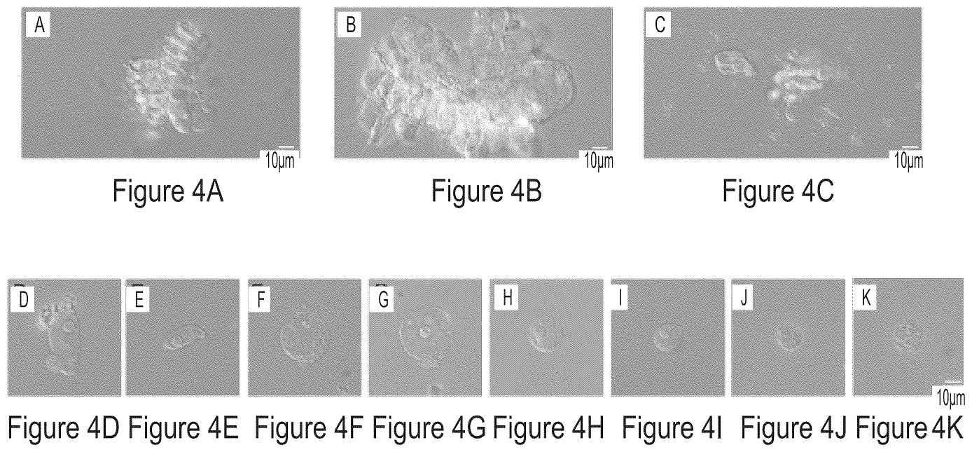

To date, >200 OHCs, >200 IHCs, and >200 Deiters cells have been isolated across multiple time points including P15, P30, P60 and P228 using the Single-Cell Dissection Technique. Each cell is classified based on its morphology and photographed as part of the collection process (FIGS. 1A-1B). RNA-Seq data from these cells have confirmed the observations made by Rehman and colleagues but also identify further transcriptional complexity of MYO15A that was not recognized (FIGS. 2A-2G). Single cells are isolated from the cochlea at multiple time points (initially focusing on P15, P60 and P228) (FIGS. 3A-3D). Frequently seen cell types are shown in FIGS. 4A-4K.

Murine Inner Ear Dissection: To identify and classify novel exons and inner-ear specific isoforms of candidate genes, cochlear tissue is harvest from male and female C3-Heb/FeJ and C57BL/6 mice; to identify and quantitate pathway changes associated with specific genetic mutations in mouse models of hearing loss, cochlear tissue from male and female Beethoven (Bth) mice (Tmc1Bth/+ and Tmc1Bth/Bth), Kncq4+/− mice, and wild-type C57BL/6 and C57BL/6 Cdh23c.753A>G mice are harvested. The single cell isolation procedure follows the general method described in Liu et al 2014, although we have introduced key modifications to optimize downstream single-cell RNA-seq (Liu H, Pecka JL, Zhang Q, Soukup GA, Beisel KW, He DZ. Characterization of transcriptomes of cochlear inner and outer hair cells. J Neurosci 2014 August;34 (33): 11085-95. doi: 10.1523/JNEUROSCI.1690-14.2014. PMID: 25122905).

Extracting the Membranous Labyrinth: Mouse cochleae are removed from the temporal bone after euthanasia and placed in a small petri dish containing ice-cold 1×DPBS. Under a dissecting microscope (Model M165FC, Leica Microsystems, Buffalo Grove, IL), the apical portion of the bony labyrinth is removed using #5 forceps (Fine Science Tools, Foster City, CA). The membranous labyrinth is extracted and placed into a 1.5 mL tube containing collagenase (Sigma Aldrich) and 1×DPBS at a working concentration of 3 mg/500 μL. The tissue is allowed to digest at room temperature for 5 min and then is gently dissociated by pipetting up and down 6-8 times with a P1000 pipette. The solution is next transferred to a glass microscope slide (Superfrost Plus 25×75×1.0 mm, Fisher Scientific, Pittsburgh, PA) and placed on an inverted microscope (Model DMI3000B, Leica Microsystems, Buffalo Grove, IL) together with a separate wash slide containing a 1 mL drop of pure 1×DPBS. The microscope is equipped with a 20× and 40× DIC objective and two 3D micromanipulators (Model MN-153, Narishige, Amityville, NY), each driving a pulled glass micropipette attached to a nitrogen gas-powered Harvard Apparatus Pico-Injector (PLI-100, Harvard Apparatus, Holliston, MA).

Isolating Single Cells: The field is scanned for cells of interest. OHCs, IHCs and Deiters cells have distinct morphology that allows them to be identified easily. When a cell is selected, the glass pipette is lowered into the solution using the micromanipulator and positioned next to the cell. The operator then aspirates the cell into the pipette in a slow and controlled manner. The cell is then expelled into the 1×DPBS on the wash slide to remove unwanted contaminating mRNAs and debris. An unused clean pipette controlled by the second 3D micromanipulator is used to re-aspirate the desired cell, which is placed into a 0.2 mL tube containing lysis buffer, RNase inhibitor and ERCC controls. The lysed samples are stored on ice until all isolations are completed.

Library Preparation, Sequencing: Library preparation and reverse transcription are performed immediately following single-cell isolations as described by Picelli et al 2014, with several minor differences (Picelli S, Faridani OR, Björklund AK, Winberg G, Sagasser S, Sandberg R. Full-length RNA-seq from single cells using smart-seq2. Nat Protoc 2014 January;9 (1): 171-81. doi: 10.1038/nprot.2014.006. PMID: 24385147). For reverse transcription, the Superscript III Reverse Transcriptase (RT) kit is used rather than the Superscript II RT kit. Also the number of cycles in the PCR pre-amplification step is increased from 19 to 22 cycles, and in the step to amplify the adapter-ligated fragments, 14 cycles are used instead of 6-8. The tagmentation and amplification-of-adapter-ligated-fragments reactions are carried out using ½ volume reactions. Pooled libraries are sequenced on a single lane of an Illumina HiSeq 4000.

### Example 2

### Feasibility of RNAi as a Broadly Applicable Tool to Prevent and Reverse Genetic Hearing Loss

The present experiments in mouse models of ADNSHL using RNAi at different time points after the onset of hearing loss to determine investigate whether a ‘window of therapeutic opportunity’ exists during which hearing loss is reversible. It is hypothesized that there is a period of time after the onset of hearing loss in the Beethoven (Bth) mouse during which the loss is not permanent. By suppressing the endogenous deafness-causing Bth allele during this ‘time window,’ the hearing loss can be reversed by RNAi-based gene therapy. Beyond this ‘time window,’ it is hypothesized that the hearing loss will continue irrespective of the ability to suppress the Bth allele. To test this hypothesis, artificial miRNA that was validated in Bth mice treated at P0-2 is used treat animals over a range of time points initially focusing on P15 and P60.

In the present methods, RNAi is used to silence both alleles in mouse models of hearing loss, with concomitant gene replacement using an appropriately engineered exogenous wild-type allele. In this example, it is shown that RNAi can effectively suppress the expression of both endogenous alleles in the Tmc1Bth/+ mouse (a model of ADNSHL) and the Tmc1Bth/Bth mouse (a model of ARNSHL), with rescue of hearing achieved by co-delivery of an exogenous wild-type Tmc1 allele engineered to resist suppression by the selected artificial miRNA. This type of ‘gene-based’ targeting strategy offers two potential advantages over a ‘mutation-based’ targeting strategy. First, it obviates the need to design literally thousands of ‘mutation-specific’ types of gene therapy, and second, it means that one RNAi-based construct can be used to treat all types of hearing loss (ADNSHL and ARNSHL) associated with a given gene.

### BACKGROUND

A number of approaches have been studied to assess the potential promise of gene therapy as a treatment for hearing loss. Recent successes have been reported using antisense oligonucleotides and gene replacement therapy. A third approach is the use of RNAi to selectively suppress the mutant allele in mouse models of ADNSHL. It has been shown that in the Beethoven mouse (Tmc1Bth/+) a single injection of an appropriately designed artificial miRNA can suppress the endogenous deafness-causing allele and preserve hearing.

While mice are invaluable as an animal model in which to test gene therapy for hearing loss, the direct translation of murine results to humans is not possible. Current studies show that gene therapy does prevent hearing loss in a mouse cochlea destined genetically to fail when animals are treated at P0-2,7; however, this time point is prior to the onset of hearing making experiments in adult mice highly germane. Unfortunately, heretofore, reliable transduction of cochlear hair cells has not been possible in the adult mouse. To address this challenge, a robust surgical approach has been developed that allows the transduction of hair cells in any animal at any age.

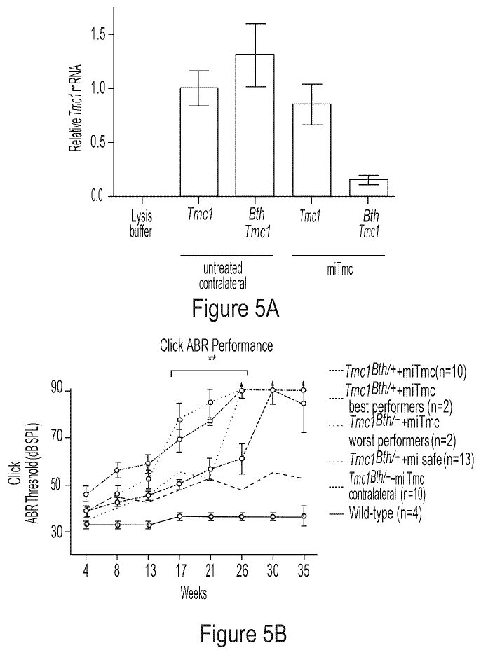

Using the Beethoven mouse, numerous artificial miRNAs were screened to identify one that could selectively and specifically suppress the mutant Bth allele. The efficacy of the selected miRNA was evaluated in vivo by collecting hair cells 4 weeks after surgery and documenting selective suppression of the deafness-causing Bth allele (FIG. 5A). Based on these findings a longitudinal study was completed in Beethoven mice to determine the effect of miRNA-based gene therapy on the hearing loss phenotype.

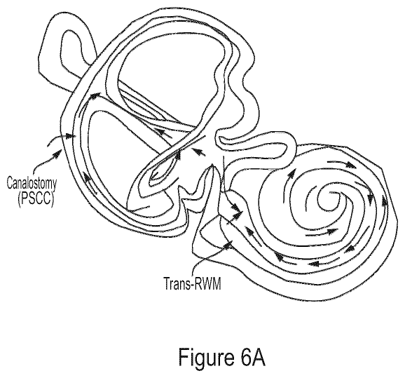

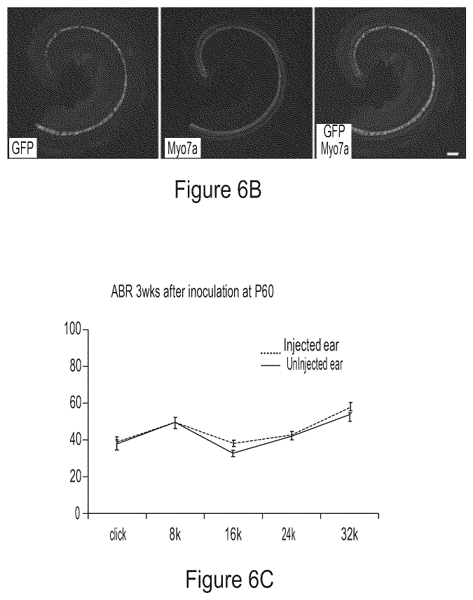

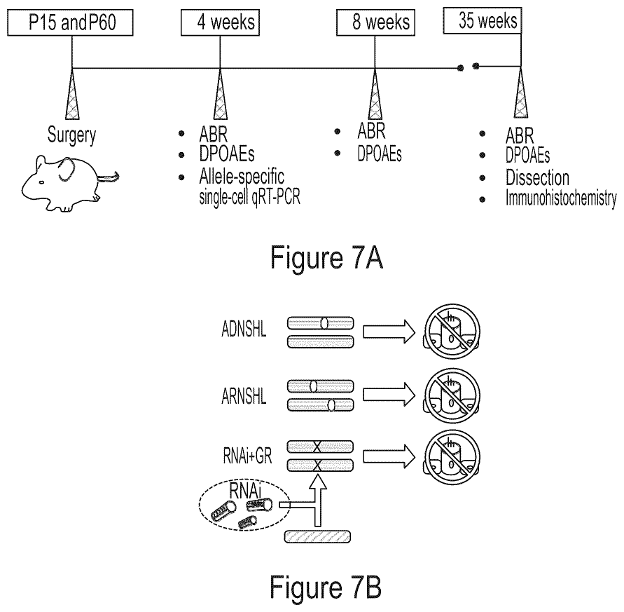

In all treated animals, asignificant preservation of hearing was found as compared to controls, although the duration of this effect was only 21 weeks. By 30 weeks post-injection, most treated animals had increased ABR thresholds; however, in the two best performing animals, hearing thresholds remained stable at ˜15-20 dB above thresholds for wild-type C3HeB/FeJ litter-mate controls for the entire study period (dashed line in FIG. 5B). A robust surgical approach has been developed that allows transduction of hair cells in adult animals (FIGS. 6A-6C). A general overview of the experiments is shown in FIGS. 7A-7B.

Mice: Mice are housed in a controlled temperature environment on a 12-hour light/dark cycle with food and water provided ad libitum. An isogenic heterozygous strain of Beethoven mice (Tmc1Bth/+) on a C3HeB/FeJ (C3H) background is maintained. Genotyping is done on DNA extracted from tail clips using phenol/chloroform and then amplified with forward and reverse primers in a 25 μl reaction volume containing 150 ng DNA, 0.2 nM of each primer and BioLase DNA polymerase (Bioline USA Inc, Taunton, MA) to generate a 376-bp amplification product in Tmc1Bth/+ mice, as described (Shibata SB, Ranum PT, Moteki H, Pan B, Goodwin AT, Goodman SS, Abbas PJ, Holt JR, Smith RJH. RNA interference prevents autosomal dominant hearing loss. Am J Hum Genet 2016 May;98:1101-13. doi: 0.1016/j.ajhg.2016.03.028. PMID: 27236922).

Design of RNAi Constructs and Engineering of Wild-type Alleles: siRNA sequence design is performed using siSPOTR to identify potent candidate siRNA sequences that target Tmc1 with low potential for off-target effects. The top ˜12 candidate siRNA sequences aree cloned into mU6-driven miRNA expression plasmids containing a CMV-driven eGFP marker and individually transfected in biological triplicate into COS7 cells (which do not contain native Tmc1) alongside a plasmid expressing wild-type Tmc1. 24 hr post-transfection, GFP expression is evaluated as an indicator of transfection performance. RNA is extracted from transfected cells with Trizol reagent, DNase treated, quantified and normalized prior to first-strand reverse transcription using SuperScript® III (Thermo-Fisher Scientific). The resulting cDNA is used for SYBR® Green-based qPCR analysis to assess performance of each siRNA design in biological triplicate and technical triplicate (a total of 9 qPCR reactions per siRNA design). qPCR results are quantified using the ΔΔCT method (see FIGS. 5A-5B).

Next, multiple synonymous nucleotide changes (ideally >3) at the siRNA recognition site for each of the selected siRNAs are introduced into murine wild-type Tmc1 constructs using site-directed mutagenesis. Three different engineered Tmc1 constructs Tmc1eng1-3 are cloned into plasmids and co-transfected into COS7 cells with the appropriate mU6-driven miRNA expression plasmid. The expression studies described above are repeated and the siRNA showing the least suppression of its corresponding Tmc1eng construct is carried forward for further study.

Virus Production: miRNA expression plasmids are altered to replace the CMV-driven eGFP construct with the engineered copy of the murine Tmc1eng and packaged into AAV vectors using a standard triple-transfection method in 293 FT cells. Viral particles are isolated by purification on a cesium chloride gradient. Constructs are packaged into two viral serotypes, AAV2/9 and AAV/Anc80, both of which robustly transduce IHCs and OHCs in mice of all ages. Control constructs (miSafe in place of the selected miRNAs; CMV-eGFP in place of Tmc1-eng) are produced and packaged into the same viral serotypes.

Viral Inoculation: Viral inoculation is performed through the round window membrane (RWM) after completing a canalostomy. Using this method, robust transduction of cochlear tissue is possible in animals of any age (FIGS. 6A-6C). In brief, animals are anesthetized with an intra-peritoneal injection of ketamine (100 mg/kg) and xylazine (10 mg/kg), body temperature is maintained with a heating pad, and the left post-auricular area is shaved and cleaned. Under an operating microscope, a post-auricular incision is made and the facial nerve is identified deep along the wall of the external auditory canal. The cochlea bulla is ventral to the facial nerve and is entered by making a hole with a 0.5 mm otologic drill, which is widened to visualize the stapedial artery and the RWM. The 0.5 mm otologic drill is also used to make a hole in the posterior semicircular canal, which is dorsal to the cochlea bulla. Once efflux of fluid from the canalostomy is seen, attention is redirected to the RWM. 2 μl of AAV with 2.5% fast green dye is loaded into a borosilicate glass pipette (1.5 mm OD×0.86 mm ID, Harvard Apparatus) pulled with a Sutter P-97 micropipette puller to a final OD of ˜20 μm and affixed to an automated injection system pressured by compressed gas (Harvard Apparatus). Pipettes are manually controlled with a micropipette manipulator. The RWM is punctured gently in the center and AAV is slowly microinjected into the scala tympani over 120 sec. Efflux of green fluid from the canalostomy indicates a successful inoculation. The RW niche is then sealed with a small plug of muscle to prevent leakage from the RWM and the bony defects of the canal and bulla are closed using small plugs of muscles. The incision is closed in layers using interrupted sutures.

### Auditory Testing

Auditory Brainstem Response (ABR): Mice are anesthetized using ketamine and xylazine at 100 mg and 6 mg per kg body weight, respectively. ABR thresholds are obtained for both clicks and tone bursts. Recordings are made from both ears of all animals in a sound-attenuating room.

Distortion Product Otoacoustic Emissions (DPOAEs): DPOAEs at (2f1-f2) are measured using f2 frequencies from 4 to 32 kHz in ½-octave steps, with f2/f1=1.22. The levels of the primaries are fixed at 65 dB SPL and 55 dB SPL for f1 and f2, respectively. For each f2 frequency, 10 1-second stimulus presentations will be averaged. DPOAE amplitudes and associated noise floors are calculated from FFT analysis of the averaged waveforms.

Immunohistochemistry and Histology: In brief, injected and non-injected cochleae are harvested after sacrificing animals using CO2 inhalation. Temporal bones are removed, perfused with 4% paraformaldehyde, incubated for 1 hr, and then rinsed in PBS and stored at 4° C. in preparation for dissection and immunohistochemistry. Specimens are infiltrated with 0.3% Triton X-100 and blocked with 5% normal goat serum prior to a 1 hr incubation with rabbit polyclonal Myosin-VIIA antibody (Proteus Biosciences Inc, Ramona, CA) or mouse monoclonal antibody to GFP (Millipore, Temecula, CA) diluted 1:1000 in PBS. A 30 min incubation in a 1:1000 dilution of the secondary antibody (fluorescence-labeled anti-rabbit IgG Alexa Fluor 568 or goat anti-mouse IgG Alexa Flour 488; Invitrogen, Eugene, OR) follows. Filamentous actin is labeled by a 30 min incubation of phalloidin conjugated to Alexa Fluor 488 (Invitrogen, Eugene, OR).

Specimens are mounted in diamond mounting medium (Life Technologies, Carlsbad, CA), and Z-stack images of whole mounts will be collected at 10-40× on a Leica SP8 confocal microscope (Leica Microsystems Inc, Bannockburn, IL). Maximum intensity projections of Z-stacks are generated for each field of view and composite images showing the whole cochlea will be constructed in Adobe Photoshop CS6, measuring distance from the apex in 0.25 mm or 40 mm increments using imageJ (NIH Image). IHC and OHC survival is quantitated using 20×-40× images of whole-mount cochleae compiled into cochleograms at 35 weeks.

Molecular Studies of In Vitro and In Vivo Expression: The constructs are validated by completing in vitro miRNA screening using COS-7 cells grown in DMEM (Invitrogen, Waltham, MA) with 10% fetal bovine serum at 37° C. and 5% CO₂. The transfection mix is made using Lipofectamine 2000 according to the manufacturer's protocol (Invitrogen, Waltham, MA). miRNA expression plasmids will be cotransfected with p.AcGFPmTmc1ex1Bth. RNA is extracted from cells using TRIzol® (Invitrogen, Waltham, MA) and expression levels are assessed in triplicate by RT-PCR (StepOne Plus, ABI), normalizing results to beta-actin. Controls include U6 miSafe and empty vector. miRNA expression plasmids with >50% suppression are cotransfected with p.AcGFPmTmc1ex1wild-type and p. AcGFPmTmc1ex1wild-type [engineered] to assess co-suppression of the normal wild-type allele and absence of suppression of the modified wild-type allele that has been engineered to resist the miRNA.

For in vivo expression analysis, the left ear of Tmc 1Bth/+ and Tmc1Bth/Bth mice is injected with AAV2/9 or Anc80 carrying the appropriate miTmc construct for SA2a or SA2b; the right ear serves as a non-injected control. Cochleae are harvested 28 days post-injection and Tmc1 expression is quantitated using appropriate allele-specific primers, amplifying each sample in triplicate. Results are normalized to β-actin expression using the ddCt algorithm. The studies begin on P0-2 animals (FIG. 7B), moving to other time points.

Statistical Analysis: Statistical analysis of ABR, DPOAE and cell counting data are completed in R using two-sample t-tests for samples of equal variance. Samples with unequal variance are compared using Welch two-sample T-tests. Sample variance is determined using the F test to compare two variances.

### Example 3

Intravenous rAAV2/9 Injection for Murine Cochlear Gene Delivery

Gene therapy for genetic deafness is a promising approach by which to prevent hearing loss or to restore hearing after loss has occurred. Although a variety of direct approaches to the inner ear have been described, presumed physiological barriers have heretofore precluded investigation of systemic gene delivery to the cochlea. In this study, we sought to characterize systemic delivery of an rAAV2/9 vector as a non-invasive means of cochlear transduction. In wild-type neonatal mice (post-natal day 0-1), we show that intravenous injection of rAAV2/9 carrying an eGFP-reporter gene results in binaural transduction of spiral ganglion neurons, inner and outer hair cells, the stria vascularis and vestibular organs. Transduction efficiency increases in a dose-dependent manner. Inner hair cells are transduced in an apex-to-base gradient, with transduction reaching 96% in the apical turn. Hearing acuity in treated animals is unaltered at postnatal day 30. Transduction is influenced by viral serotype and age at injection, with less efficient cochlear transduction observed with systemic delivery of rAAV2/1 and in juvenile mice with rAAV2/9. Collectively, these data validate intravenous delivery of rAAV2/9 as a novel and atraumatic technique for inner ear transgene delivery.

### INTRODUCTION

Hearing loss is the most common sensory impairment in humans. It impacts 1 of every 1000 newborns and in 70% of these babies has an underlying genetic etiology¹. Current clinical treatment options for hereditary hearing loss are limited to sound amplification and cochlear implantation². Although these interventions are nearly always beneficial, when compared to biological hearing performance outcomes are modest. To preserve biological hearing, targeted or personalized habilitation options that focus on preserving or even restoring hearing function by inner ear transgene delivery have gained interest.

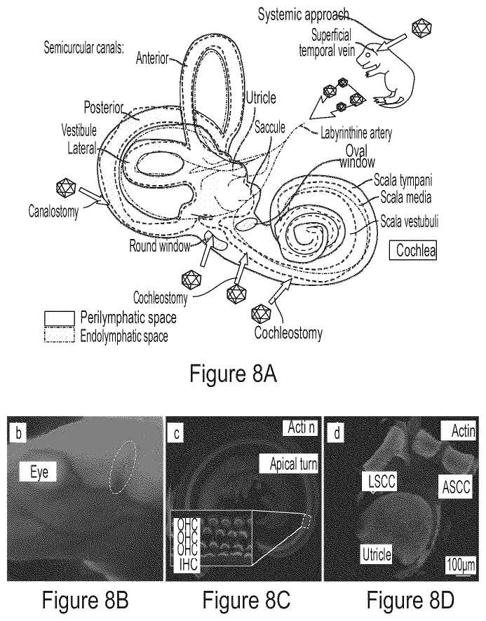

One of the hurdles facing cochlear gene transfer is the delivery of a safe yet efficient amount of therapeutic to the cochlear epithelium. Because the mammalian inner ear is encased in the temporal bone, direct surgical intervention to access the membranous labyrinth is not trivial and can lead to unwanted side effects³⁻⁵. Established approaches to the perilymphatic or endolymphatic compartments include: (a) the perilymphatic approach, via a trans-round window membrane (RWM) injection⁶⁻⁸, cochleostomy to the scala tympani^{9,10}, or semicircular canal canalostomy¹¹⁻¹³ and (b) the endolymphatic approach, with a direct cochleostomy to the scala media^{14,15} (FIG. 8A). While the perilymphatic approach is relatively safe and commonly used for cochlear implantation in humans¹⁶, the endolymphatic approach is complex and carries a high risk of inner ear damage making it clinically unfeasible, although efforts are on-going to establish an atraumatic approach to the endolymphatic space in neonatal and adult murine models^{15,17}. The development of a non-invasive and non-surgical method of transducing the inner ear may drive the translation of cochlear gene transfer into clinical practice.

To our knowledge, systemic gene transfer targeting the sensory and non-sensory epithelium in the inner ear has not been attempted (FIG. 8A). Reasons for this omission include potential systemic toxicities and two physiological barriers: the blood-brain barrier (BBB) and blood-labyrinth barrier (BLB), which obscure attempts to deliver larger molecules from the circulation into the target cells. The BBB is formed by endothelial tight junctions, pericytes, astrocytes and cellular basement membranes. Together, these structures comprise a barrier that precludes the entry of >98% of small molecules and most macromolecules¹⁸. The BLB has similar cellular structure and provides selective permeability by which to maintain inner ear homeostasis.

The recent finding that recombinant adeno-associated virus (rAAV) serotype 2/9 crosses the BBB after intravascular injection in postnatal and adult mice has impacted gene therapy studies targeting the CNS and retina¹⁹⁻²¹. For example, systemic gene therapy using rAAV2/9 is effective as a treatment for spinal muscular atrophy in P1-2 mice²². rAAV serotypes 2/1, 2/6, 2/7 and 2/10 also cross the BBB, however their CNS tissue transduction characteristics vary²³. Other AAV serotypes, such as 2/2, 2/5 and 2/8, are BBB impermeable, although the precise mechanism for these differences is unknown²⁴.

Direct surgical approaches have been used to characterize the transduction profiles of rAAV2/1 and 2/9 in neonatal/adult mouse and guinea pig cochleae however the ability of these vectors to traverse the BLB and transduce sensory and/or non-sensory cells in the inner ear epithelium has not been tested. Establishing the potential for systemic therapy is germane as there is increasing interest in delivering early post-natal preventative therapy to rescue hereditary deafness in mutant mice (specifically 0 to 48 hours after birth). In a recent proof-of-principle study, successful auditory restoration was achieved in mice with a targeted deletion of VGLUT3 following neonatal trans-round window membrane injection of AAV1 carrying VGLUT3²⁵ Hair cell transduction was higher and auditory restoration lasted longer when treatment was initiated at an earlier time point (post-natal day 1-2, P1-2) as compared to a later time point (P10)²⁵. Successes have also been reported with early interventions using antisense oligonucleotide and RNA interference to rescue or prevent hearing loss in mouse models of genetic deafness^{26,27}. Enhanced delivery of therapeutics to target cells of the neonatal ear is expected to result in even better performance outcomes.

In this study, we evaluated the transduction profile and efficiency of systemically introduced rAAV2/9 vectors tagged with eGFP as a reporter gene in wild-type neonatal murine ears. Our results indicate that rAAV2/9 transduces auditory sensory epithelium in a binaural dose-dependent fashion without affecting auditory thresholds. Inner ear transduction was less robust in neonatal mice receiving intravenous rAAV2/1 and in juvenile mice receiving rAAV2/9. These results suggest that intravenous injection of rAAV2/9 can be used in neonatal mice as an atraumatic and relatively simple method to deliver gene therapy to the cochlea.

### Results

Intravenous Delivery of rAAV2/9-CMV-eGFP Leads to Robust Dose-Dependent Transgene Expression in Neonatal Ears

To investigate the inner transduction profile following intravenous injection of rAAV2/9-CMV-eGFP in neonatal mice, and whether transduction efficiency could be improved in a dose-dependent manner, intravascular injections were performed via the superficial temporal vein delivering a total volume of 50 μl to neonatal mice (FIG. 8B). Two different concentrations of rAAV2/9-CMV-eGFP were administered: either 3.28×10¹³ (high titer) or 6.55×10¹² (low titer; 1/5 of the high titer) vg/ml.

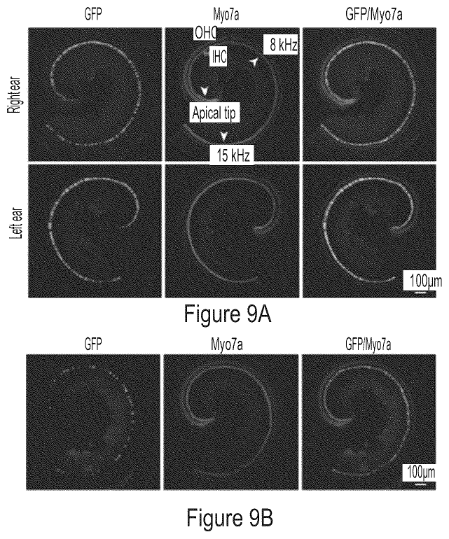

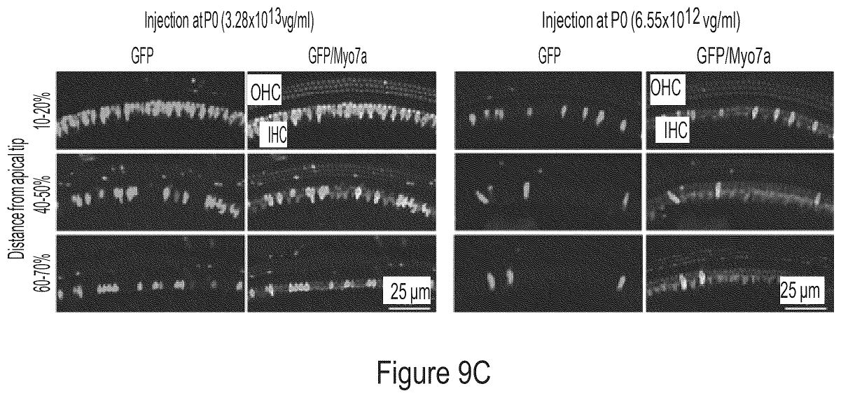

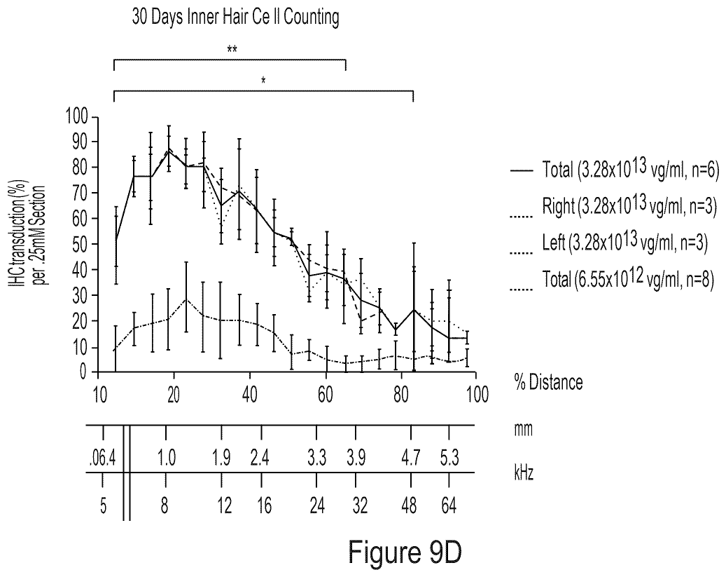

Thirty days after delivery of rAAV2/9-CMV-eGFP, whole mount sections of the membranous labyrinth were analyzed to quantitate inner hair cell transduction (FIGS. 8C and 8D). All injected mice demonstrated a similar transduction profile, with inner hair cells (IHCs) being the primary cell type transduced (FIGS. 9A-9C). All mice demonstrated strikingly similar binaural inner ear eGFP expression (FIG. 9a), with an obvious dose-dependent effect (FIGS. 9b and 9V). The distribution of the eGFP was more robust in the apical as compared to the basal turn of the cochlea (FIG. 9C). A cochleogram plotted along the length of the cochlear duct showed that mice receiving the higher titer had significantly greater transduction of IHCs in the apical turns, with up to 96% wene transduction 1 mm distal to the apex (FIG. 9D). IHC transduction decreased in apex-to-base gradient along the length of the cochlear duct. Of note, there was no hair cell loss associated the injection.

Spiral Ganglion Cells and the Stria Vascularis are Transduced Following rAAV2/9-CMV-eGFP IV Injection

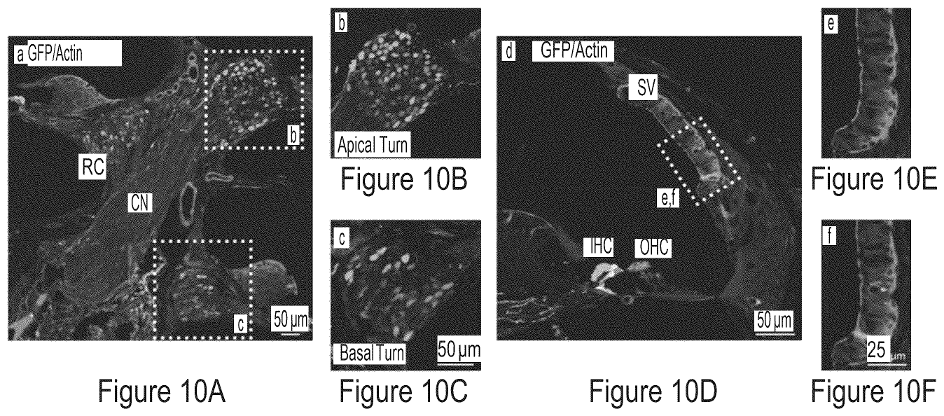

To define the extent of transduction of spiral ganglion cells and stria vascularis, we analyzed cochlear frozen cross sections in mice treated with the higher titer of rAAV2/9-CMV-eGFP. Robust transduction in the soma of the spiral ganglion cells was observed, with more prominent expression in the apical as compared to basal turns (FIGS. 10A-10C). Transduction of the nerve fibers of the bipolar cells was also noted. Expression of eGFP was observed in the capillary vessels and adjacent fibrocytes of the stria vascularis (FIGS. 10D-10F).

Vestibular Organs are Transduced Following rAAV2/9-CMV-eGFP IV Injection

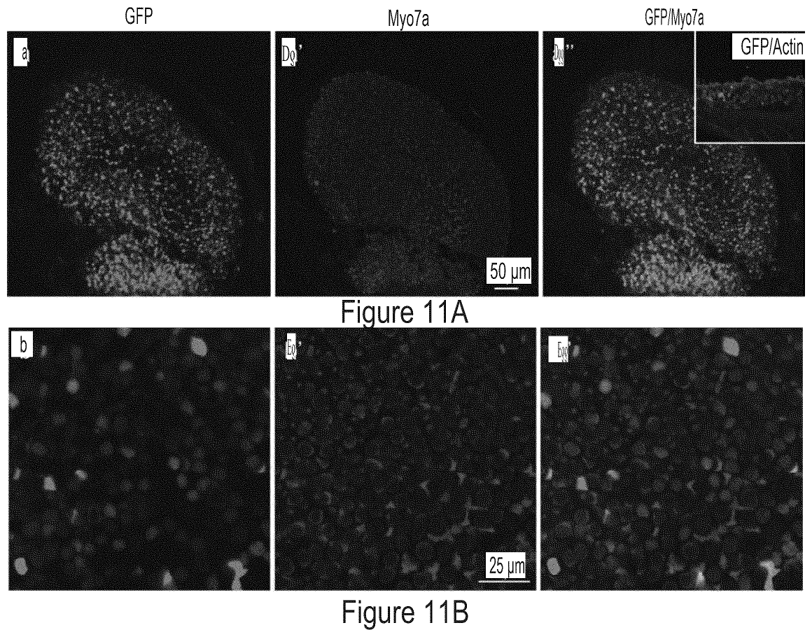

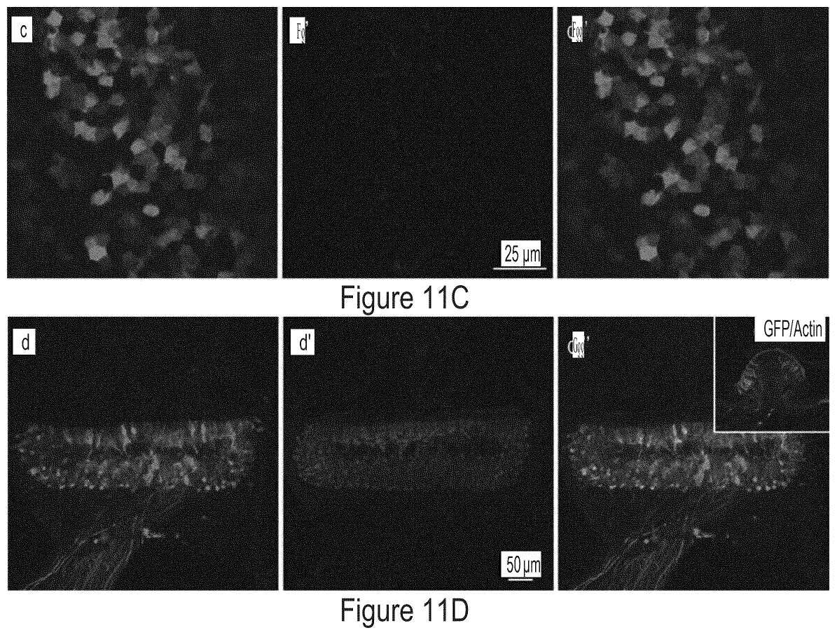

The vestibular organ is an equally important target for inner ear gene transfer and shares equal vasculature with the cochlea. We investigated transgene expression in the utricle and ampullaris of the anterior semicircular canal on whole mount and frozen sections (FIG. 8C). Robust transduction in both utricles (FIG. 11a-a″) and ampullae (FIG. 1d-d″) was observed. In the utricles, the vestibular hair cells (FIG. 11b-b″) and underlying vestibular supporting cells (FIG. 11c-c″) demonstrated eGFP transgene expression. In the ampullae, eGFP expression was noted in the vestibular hair cells and vestibular nerve fibers. Treated mice did not demonstrate circling or head tilting.

Auditory Thresholds are Unchanged by Neonatal IV rAAV2/9-CMV-eGFP Injection

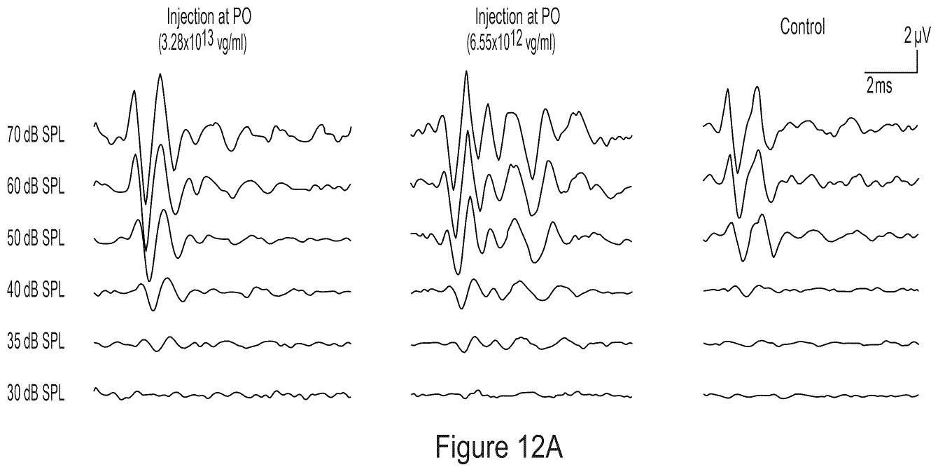

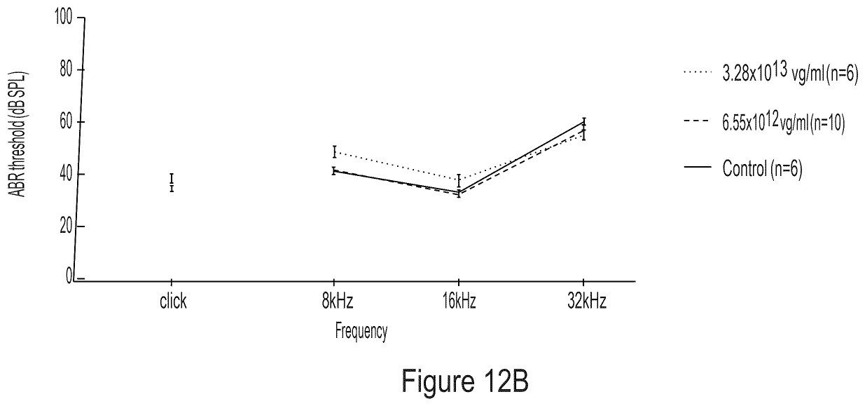

We assessed auditory function by measuring auditory brainstem response (ABR) thresholds 4 weeks after intravenous injection to assess potential ototoxicity. Bilateral ears were measured in all animals from each group and in non-injected control animals. There were no statistically significant differences between ears in treated animals, and auditory performance in all treatment groups was comparable to the untreated control group (FIGS. 12A and 12B). These results suggest that intravenous injection and IHC transduction does not alter auditory function. Likewise, treated mice did not demonstrate behavioral side effects (e.g. head tilting, weight loss, circling or ear infection) and were healthy.

### Inner Ear Transduction Following IV Injection is AAV Serotype Dependent

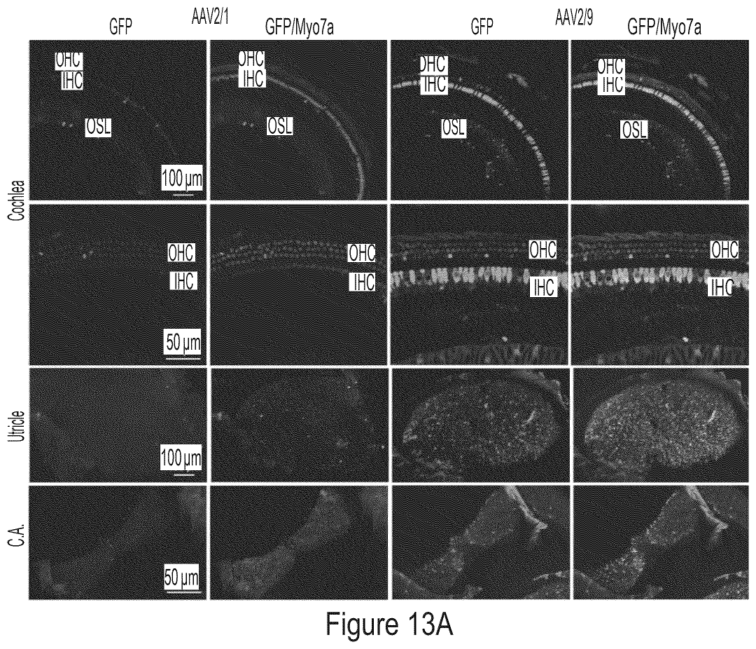

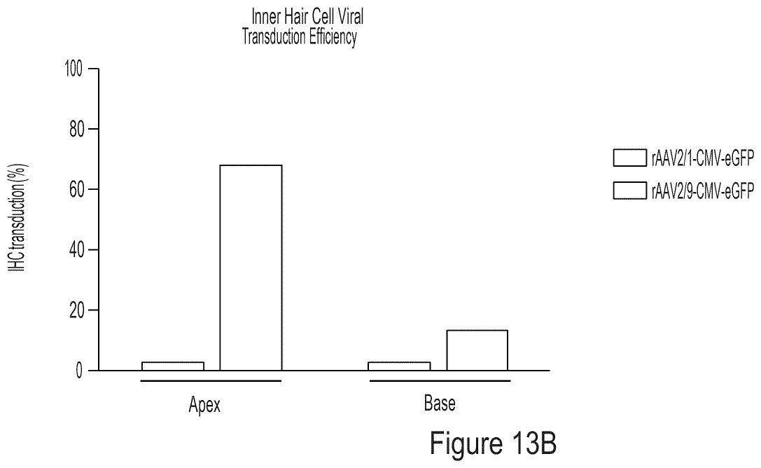

We compared intravenous injection of rAAV2/1 and 2/9 using titers of 3.09×10¹² vg/ml and 1.59×10¹² vg/ml, respectively. With rAAV2/9-CMV-eGFP, expression of eGFP was observed in both cochlear and vestibular tissues (FIG. 13A). IHCs in apical half turns of the membranous labyrinth were primarily transduced, with sparse transduction of OHCs and supporting cells. Vestibular hair cells in the utricle and ampullaris of the anterior semicircular canal were also transduced. In comparison, when intravenous rAAV2/1-CMV-eGFP was used, expression was restricted to a few hair cells and supporting cells in both the membranous labyrinth and the vestibular organs suggesting that transduction efficiency is serotype dependent and that rAAV2/9 is superior to rAVV2/1 (FIG. 13B).

Decreased Inner Ear Gene Expression Following IV Injection of rAAV2/9-CMV-eGFP in Juvenile Ages

We investigated the temporal window of efficient inner ear transduction by injecting 100 μl of rAAV2/9-CMV-eGFP at 3.28×10¹³ vg/ml in juvenile mice at P14-15. Because use of the superficial temporal vein is not feasible at this age, injections were completed via the external jugular vein. Ears were harvested at P30 and dissected for whole mount preparation. We found very limited transduction with few countable eGFP-positive hair cells in the apical and basal turns. While the presence of eGFPpositive cells demonstrates the feasibility of inner ear transduction at this age, efficiency with rAAV2/9 is markedly reduced.

Wide-Spread Transduction of the Brain and Skeletal Muscles Follows rAAV2/9-CMVeGFP IV Injection

As systemic injection of rAAV2/9 facilitates wide-spread transduction, we sought to examine the transduction profile in other organs. Gene expression was observed in the cerebral cortex and cerebellum, and in the skeletal muscle of the quadriceps, consistent with reported observations when rAAV2/9-CMV-eGFP is used for other purposes^{19,20,28}. We found that astrocytes and Purkinje cells were transduced with higher efficiencies in neonatal mice receiving higher doses. Mice injected via the external jugular vein at P14-15 showed similar transduction profiles although efficiency was limited, similar to the changes we observed in the inner ear and again consistent with other studies²⁸. In contrast, transduction of muscle was stable in both neonatal and juvenile mice.

### DISCUSSION

This study is the first to validate intravenous injection as a systemic approach for cochlear gene delivery. We show that if rAAV2/9 is used, widespread binaural transduction can be mediated in the cochlea, spiral ganglion, stria vascularis and vestibular organs. The technique is safe and does not affect auditory thresholds, although there are age- and viral serotype-dependent effects. It is important to note that the procedure should be broadly applicable for cochlear gene therapy in neonatal mice with various forms of congenital deafness or vestibular dysfunction. Since the first reported use of rAAVs for in vivo cochlear gene transfer experiments 20 years ago²⁹, multiple inoculation methods have been developed to maximize efficiency and minimize iatrogenic trauma. In this study, we have shown that the intravascular injection of rAAV2/9 offers a simple and atraumatic method to deliver transgenes to the neonatal inner ear. In comparison to other delivery methods, this approach is: (1) very simple, although practice is required to successfully inject the superficial temporal vein; (2) inexpensive, requiring only a basic set up for an intravenous injection; (3) atraumatic, as the ear is not accessed; (4) binaural, offering simultaneous transduction of both the membranous cochlea and vestibular organs; (5) reproducible; and (6) potentially applicable to mice of all ages (including fetal mice via the yolk sac vein²⁰). Disadvantages of this method include: (1) global transduction, which leads to transgene expression in off-target tissues that may result in off-target side-effects; (2) volume limitations, which should not exceed 100 μl in neonates to avoid hypervolemia; (3) transduction limitations, both in terms of cell type, cell location and animal age, which may reflect viral tropism of rAAV2/9; and (4) delivery confirmation, as successful delivery to the ear at the time of injection cannot be confirmed.

The potential feasibility of intravascular injection of AAVs for cochlear gene transfer is not intuitive. The cochlea is separated by a tight BLB, which shares many similarities with the BBB^{30,31}. It prevents molecules and viruses from entering the endolymphatic and perilymphatic spaces. Nevertheless, our data show that rAAV2/1 and 2/9 reach the cochlear duct (FIG. 13A). The presence of eGFP expression in the stria vascularis and spiral limbus suggests that rAAV vectors may extravasate from the vasculature and enter the cochlear duct lumen by permeation of the BLB (FIG. 10D). The exact mechanism of AAV2/9 crossing BBB into the CNS is unknown although transcytosis is thought to be associated with the crossing the endothelial barrier²⁴. Further investigation is needed to determine whether these entry pathways/transport mechanisms are used in the cochlea. We found that intravenous injection in juvenile mice provides only limited inner ear transduction, although transgene expression remains stable in tissues like muscle and cerebellum. This observation is consistent with other reports demonstrating that the expression pattern of rAAV2/9 following systemic injection is influenced by age and that maturation of the BBB leads to a narrow therapeutic window for tissues like the retina³². Thus while the BLB reduces viral entry into membranous labyrinth in adult mice, it is possible that entry can be potentiated. For example, hyperosmolar mannitol pretreatment improves CNS transduction of AAV2 and AAVrh10 vectors by temporarily disrupting the BBB^{33,34}, although rAAV2/9 transduction is not impacted²⁸. Testing various rAAV serotypes and the effect of mannitol pretreatment may widen the window for intravenous delivery of therapeutics to the inner ear.

Currently, the perilymphatic approach is used most commonly to deliver vectors into the inner ear. It is minimally invasive injection and has been clinically established for cochlear implantation. As compared to systemic injection, its main advantage is high target selectivity, which minimizes viral spread and limits transgene expression to the treated ear. Thus from a safety perspective, perilymphatic injection will likely remain the favored approach when gene-therapy-based clinical trials begin. However, relatively low transduction in the inner ear following perilymph injections has been a challenge. Recent emergence of engineered synthetic AAV vectors, including exosome-associated AAV (Exo-AAV) 35 and ‘designer’ ancestral aav2/anc80^{13,36} have boosted gene transduction in the inner ear, with up to 100% transduction of IHCs in neonatal. However, the risk of iatrogenic trauma during inner ear surgery remains a concern particularly in protecting intact functioning hair cells.

Our study demonstrates the feasibility of cochlear gene delivery without the necessity for direct inner ear access. In an earlier study, we showed that rAAV2/9 has a predilection for transduction of apical IHCs when injected through the round window membrane; in this study we have shown that intravenous injection provides comparable IHC transduction efficiency²⁷. These similarities suggest a tropism of rAAV2/9 for apical IHCs, which is not impacted by injection method. Importantly, systemic injection make transduction of spiral ganglion cells and vestibular hair cells feasible. From a technical perspective, the systemic method is simple compared to perilymphatic and endolymphatic approaches, and can be implemented without purchasing specialized tools like micropipettes, micromanipulators and nanoinjection systems. The global transduction associated with systemic delivery of rAAV2/9 may make this method applicable to syndromic types of hearing loss, like hereditary keratitis-ichthyosisdeafness syndrome and the Usher syndromes³⁷. This approach also may be useful when hearing loss is secondary to metabolic or neurodegenerative disorders, as systemic therapy could conceivably correct both the hearing loss and its underlying cause. Although unwanted off-target effects or immunological side effects raise safety concerns, the use of tissue-specific promoters such as Myo7a38, Pou4F3 39 and the 9 subunit of the acetylcholine receptor 40 may obviate these concerns.

In conclusion, this study validates intravenous injection as a systemic approach for cochlear gene delivery and shows that widespread binaural transduction is possible. The technique is easy and safe, and may be widely applicable for cochlear gene therapy in neonatal mice with various forms of congenital deafness. Although age- and viral serotype-dependent effects are seen, the advantages offered by systemic therapy justify further research to address targeted transduction and efficiency in adult cochlear tissue.

### Materials and Methods

### Virus Production

AAV viral vectors were prepared by the Gene Vector Core facility at the University of Iowa using the triple transfection method or baculovirus system as described⁴¹. Single-stranded recombinant AAV serotypes (rAAV2/1 and rAAV2/9) contained a transgene cassette of CMV-driven eGFP. For the dose-dependency study 50 μl of rAAV2/9 viral vector at 3.28×10¹³ (high titer) or 6.55×10¹² (low titer; 1/5 of the high titer) were administered. Viral titers used in intravascular injections comparison study were diluted in sterile saline to concentrations of 3.09×10¹² vg/ml for rAAV2/1 and 1.59×10¹² vg/ml for rAAV2/9. Virus aliquots were stored at −80° C. and thawed before use.

### Animal Model and Viral Inoculation

Murine experiments were conducted using wild-type inbred C3HeB/FeJ mice purchased from the Jackson Laboratory. Neonatal mice were operated on at P0-1. Mice were placed in a container with crushed ice for 3 to 5 minutes until the onset of hypothermal anesthesia could take effect. Intravascular injections were performed via the superior temporal vein (FIG. 8A). A total of 50 μl of each viral vector at 3.28×10¹³ (high titer) or 6.55×10¹² (low titer; 1/5 of the high titer) vg/ml with 2.5% fast green dye (Sigma-Aldrich, St. Louis, MO) was loaded into a 30-gauge syringe. Following canalization of the vein, the viral vector was slowly injected; upon successful injection mice turned green almost immediately. After the injection, neonatal mice were placed on a heating pad for recovery and rubbed with bedding before being returned to their mother⁴². Mice at P14 were anesthetized with intraperitoneal ketamine and xylazine at 100 mg and 10 mg per kg of body weight, respectively. They were placed in a recumbent position on a heating pad, and a small incision was made lateral to the ventral midline from the pectoral muscle to the lower neck. After the incision, an external jugular vein was exposed. Viral vectors at 3.28×10¹² vg/ml were delivered into the jugular vein using a 30-gauge needle.

### Auditory Testing

All mice were anesthetized as described above. Sound stimuli were generated using an RZ6 auditory processor driving two MF1 Multi-Field Magnetic Speakers (Tucker-Davis Technologies, Alachua, FL). Closed field transmission of the sound waves generated by the MF1 speakers was achieved by connecting 2-inch lengths of plastic tubing from the speakers to an ER-10B+ probe microphone (Etymotic Research, Elk Grove Village, IL). A speculum mounted on the ER-10B+ probe microphone was inserted into the auditory canal of the tested ear. The speculum formed a tight seal against the ear canal completing the closed field transmission of auditory stimuli. The setup was calibrated using an 0.028 cm³ cavity to approximate the size of the mouse ear canal. All recordings were conducted from both ears of all animals on a 37-degree heating pad. Clicks were square pulses 100 ms in duration and tone bursts were 3 ms in length at distinct 8, 16, and 32 kHz frequencies. ABRs were measured with BioSigRZ (Tucker-Davis Technologies) for both clicks and tone bursts, adjusting the stimulus levels in 5-decibel (dB) increments between 10-90 dB sound pressure level (SPL) in both ears. Electrical signals were averaged over 512 repetitions. ABR threshold was defined as the lowest sound level at which a reproducible waveform could be observed. Responses from wild-type inbred C3HeB/FeJ mice at 4 weeks were used as controls. ABRs were measured 4 weeks after the intravenous injection.

### Fluorescence Microscopy and Immunohistochemistry

Bilateral inner ears, brain, cerebellum and skeletal muscle were harvested 4 weeks after the intravenous injection. Deeply anesthetized animals were perfused transcardially with 4% paraformaldehyde for 15 min. Each tissue was locally perfused and fixed in 4% paraformaldehyde for 1 hr, rinsed in PBS, and stored at 4° C. in preparation for immunohistochemistry. Inner ears used in whole mount preparations for the comparison study (FIGS. 13A-13B) were stained for eGFP to enhance signal, otherwise native eGFP signal were observed. Following infiltration using 0.3% Triton X-100 for 30 min and blocking with 5% normal goat serum for 1 hr, tissues were incubated with rabbit polyclonal Myosin-VIIA antibody (Proteus Biosciences Inc., Ramona, CA) diluted 1:200 in PBS for 1 hr. Fluorescence-labeled goat anti-rabbit IgG Alexa Fluor 568 in 1:500 dilution was used as a secondary antibody (Thermo Fisher Scientific, Rockford, IL) for 30 min. For cryo-sectioning, cochleae were decalcified in 120 mM EDTA for 2 days, cryoprotected in 15% and 30% sucrose, and embedded in OCT solution. 14 μm midmodiolar cryosections were prepared and immunohistochemistry was performed. Following infiltration using 0.3% Triton X-100 for 30 min and blocking with 5% normal goat serum for 1 hr, we incubated the tissues in mouse monoclonal antibody to eGFP (Millipore, Temecula, CA) diluted 1:200 in PBS overnight. We used fluorescence-labeled goat anti-mouse IgG Alexa Fluor 488 secondary antibodies at 1:500 dilution. Filamentous actin was labeled by a 30 min incubation of phalloidin conjugated to Alexa Fluor 568 (Thermo Fisher Scientific) in 1:100 dilution (exceptions: cortex, cerebellum, skeletal muscle). Specimens were mounted in ProLong® Diamond Antifade Mounting Media (Thermo Fisher Scientific) and observed with a Leica TCS SP8 confocal microscope (Leica Microsystems Inc., Bannockburn, IL).

### Hair Cell Transduction Efficiency Analysis

Whole mount preparations as described above were used to count transduced hair cells. Images were prepared using Adobe Photoshop CC to meet equal conditions. We counted eGFP positive hair cells using the ImageJ program (NIH Image) across a 400 μm radius from the apical turn to the basal turn. Hair cells with overlapping MyoVIIA and eGFP signals were considered transduced. The total number of hair cells and eGFP-positive hair cells were summed and converted to a percentage.

### Statistical Analysis

Statistical analysis of ABR and cell-counting data was completed in R with two-sample t-tests for samples of equal variance a P value <0.05 was considered significant. Samples with unequal variance were compared with Welch two-sample t tests. Sample variance was determined with F tests comparing two variances.

### Example 3 References

1. Smith, R. J., Bale, J. F., Jr. & White, K. R. Sensorineural hearing loss in children. Lancet 365, 879-890. (2005).
2. Shibata, S. B. & Raphael, Y. Future approaches for inner ear protection and repair. J Commun Disord 43, 295-310, doi: 10.1016/j.jcomdis.2010.04.001 (2010).
3. Lalwani, A. K. & Mhatre, A. N. Cochlear gene therapy. Ear Hear 24, 342-348 (2003).
4. Hildebrand, M. S. et al. Advances in molecular and cellular therapies for hearing loss. Mol Ther 16, 224-236, doi: 6300351 [pii] 10.1038/sj.mt.6300351 (2008).
5. Shibata, S. B., Budenz, C. L., Bowling, S. A., Pfingst, B. E. & Raphael, Y. Nerve maintenance and regeneration in the damaged cochlea. Hear Res 281, 56-64, doi: 10.1016/j.heares.2011.04.019 (2011).
6. Wang, H. et al. Efficient cochlear gene transfection in guinea-pigs with adenoassociated viral vectors by partial digestion of round window membrane. Gene Ther 19, 255-263, doi: 10.1038/gt.2011.91 (2012).
7. Jero, J. et al. Cochlear gene delivery through an intact round window membrane in mouse. Hum Gene Ther 12, 539-548. (2001).
8. Qi, W. et al. Efficient siRNA transfection to the inner ear through the intact round window by a novel proteidic delivery technology in the chinchilla. Gene Ther 21, 10-18, doi: 10.1038/gt.2013.49 (2014).
9. Lalwani, A. K., Walsh, B. J., Reilly, P. G., Muzyczka, N. & Mhatre, A. N. Development of in vivo gene therapy for hearing disorders: introduction of adeno-associated virus into the cochlea of the guinea pig. Gene Therapy 3, 588-592 (1996).
10 Stover, T., Yagi, M. & Raphael, Y. Cochlear gene transfer: round window versus cochleostomy inoculation. Hear Res 136, 124-130 (1999).
11. Kawamoto, K., Oh, S. H., Kanzaki, S., Brown, N. & Raphael, Y. The functional and structural outcome of inner ear gene transfer via the vestibular and cochlear fluids in mice. Mol Ther 4, 575-585, doi: 10.1006/mthe.2001.0490 (2001).
12. Okada, H. et al. Gene transfer targeting mouse vestibule using adenovirus and adeno-associated virus vectors. Otol Neurotol 33, 655-659, doi: 10.1097/MAO.0b013e31825368d1 (2012).
13. Suzuki, J., Hashimoto, K., Xiao, R., Vandenberghe, L. H. & Liberman, M. C. Cochlear gene therapy with ancestral AAV in adult mice: complete transduction of inner hair cells without cochlear dysfunction. Scientific reports 7, 45524, doi: 10.1038/srep45524 (2017).
14. Ishimoto, S., Kawamoto, K., Kanzaki, S. & Raphael, Y. Gene transfer into supporting cells of the organ of Corti. Hear Res 173, 187-197 (2002).
15. Kilpatrick, L. A. et al. Adeno-associated virus-mediated gene delivery into the scala media of the normal and deafened adult mouse ear. Gene Ther 18, 569-578, doi: 10.1038/gt.2010.175 (2011).
16. Gantz, B. J. & Tyler, R. S. Cochlear implant comparisons. American Journal of Otology Suppl, 92-98 (1985).
17. Wang, Y. et al. Early postnatal virus inoculation into the scala media achieved extensive expression of exogenous green fluorescent protein in the inner ear and preserved auditory brainstem response thresholds. J Gene Med 15, 123-133, doi: 10.1002/jgm.2701 (2013).
18. Pardridge, W. M. Drug and gene targeting to the brain with molecular Trojan horses. Nat Rev Drug Discov 1, 131-139, doi: 10.1038/nrd725 (2002).
19. Foust, K. D. et al. Intravascular AAV9 preferentially targets neonatal neurons and adult astrocytes. Nature biotechnology 27, 59-65, doi: 10.1038/nbt.1515 (2009).
20. Rahim, A. A. et al. Intravenous administration of AAV2/9 to the fetal and neonatal mouse leads to differential targeting of CNS cell types and extensive transduction of the nervous system. FASEB journal: official publication of the Federation of American Societies for Experimental Biology 25, 3505-3518, doi: 10.1096/fj.11-182311 (2011).
21. Byrne, L. C., Lin, Y. J., Lee, T., Schaffer, D. V. & Flannery, J. G. The expression pattern of systemically injected AAV9 in the developing mouse retina is determined by age. Mol Ther 23, 290-296, doi: 10.1038/mt.2014.181 (2015).
22. Foust, K. D. et al. Rescue of the spinal muscular atrophy phenotype in a mouse model by early postnatal delivery of SMN. Nature biotechnology 28, 271-274, doi: 10.1038/nbt. 1610 (2010).
23. Zhang, H. et al. Several rAAV vectors efficiently cross the blood-brain barrier and transduce neurons and astrocytes in the neonatal mouse central nervous system. Mol Ther 19, 1440-1448, doi: 10.1038/mt.2011.98 (2011).
24. Di Pasquale, G. & Chiorini, J. A. AAV transcytosis through barrier epithelia and endothelium. Mol Ther 13, 506-516, doi: 10.1016/j.ymthe.2005.11.007 (2006).
25. Akil, O. et al. Restoration of hearing in the VGLUT3 knockout mouse using virally mediated gene therapy. Neuron 75, 283-293, doi: 10.1016/j.neuron.2012.05.019 (2012).
26. Lentz, J. J. et al. Rescue of hearing and vestibular function by antisense oligonucleotides in a mouse model of human deafness. Nat Med 19, 345-350, doi: 10.1038/nm.3106 (2013).
27. Shibata, S. B. et al. RNA Interference Prevents Autosomal-Dominant Hearing Loss. Am J Hum Genet 98, 1101-1113, doi: 10.1016/j.ajhg.2016.03.028 (2016).
28. Dufour, B. D., Smith, C. A., Clark, R. L., Walker, T. R. & McBride, J. L. Intrajugular vein delivery of AAV9-RNAi prevents neuropathological changes and weight loss in Huntington's disease mice. Mol Ther 22, 797-810, doi: 10.1038/mt.2013.289 (2014).
29. Lalwani, A. et al. Long-term in vivo cochlear transgene expression mediated by recombinant adeno-associated virus. Gene Ther 5, 277-281, doi: 10.1038/sj.gt.3300573 (1998).
30. Juhn, S. K. & Rybak, L. P. Labyrinthine barriers and cochlear homeostasis. Acta Otolaryngol 91, 529-534 (1981).
31. Juhn, S. K., Rybak, L. P. & Prado, S. Nature of blood-labyrinth barrier in experimental conditions. Ann Otol Rhinol Laryngol 90, 135-141 (1981).
32. Bostick, B., Ghosh, A., Yue, Y., Long, C. & Duan, D. Systemic AAV-9 transduction in mice is influenced by animal age but not by the route of administration. Gene Ther 14, 1605-1609, doi: 10.1038/sj.gt.3303029 (2007).
33. McCarty, D. M., DiRosario, J., Gulaid, K., Muenzer, J. & Fu, H. Mannitolfacilitated CNS entry of rAAV2 vector significantly delayed the neurological disease progression in MPS IIIB mice. Gene Ther 16, 1340-1352, doi: 10.1038/gt.2009.85 (2009).
34. Foley, C. P. et al. Intra-arterial delivery of AAV vectors to the mouse brain after mannitol mediated blood brain barrier disruption. J Control Release 196, 71-78, doi: 10.1016/j.jconrel.2014.09.018 (2014).
35. Jose, S. P. et al. Nephro-protective effect of a novel formulation of unopened coconut inflorescence sap powder on gentamicin induced renal damage by modulating oxidative stress and inflammatory markers. Biomed Pharmacother 85, 128-135, doi: 10.1016/j.biopha.2016.11.117 (2017).
36. Landegger, L. D. et al. A synthetic AAV vector enables safe and efficient gene transfer to the mammalian inner ear. Nature biotechnology 35, 280-284, doi: 10.1038/nbt.3781 (2017).
37. Schutz, M. et al. The connexin26 S17F mouse mutant represents a model for the human hereditary keratitis-ichthyosis-deafness syndrome. Hum Mol Genet 20, 28-39, doi: 10.1093/hmg/ddq429 (2011).
38. Boeda, B., Weil, D. & Petit, C. A specific promoter of the sensory cells of the inner ear defined by transgenesis. Hum Mol Genet 10, 1581-1589 (2001).
39. Vahava, O. et al. Mutation in transcription factor POU4F3 associated with inherited progressive hearing loss in humans [see comments]. Science 279, 1950-1954 (1998).
40. Elgoyhen, A. B., Johnson, D. S., Boulter, J., Vetter, D. E. & Heinemann, S. Alpha 9: an acetylcholine receptor with novel pharmacological properties expressed in rat cochlear hair cells. Cell 79, 705-715 (1994).
41. Yang, G. S. et al. Virus-mediated transduction of murine retina with adenoassociated virus: effects of viral capsid and genome size. J Virol 76, 7651-7660 (2002).
42. Gombash Lampe, S. E., Kaspar, B. K. & Foust, K. D. Intravenous injections in neonatal mice. J Vis Exp, e52037, doi: 10.3791/52037 (2014).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating hearing loss comprising:
(a) administering a gene suppression agent that suppresses both copies of an endogenous gene causing the hearing loss; and
(b) administering an exogenous wild-type allele engineered to resist suppression by the gene suppression agent.

2. The method of claim 1, wherein the gene suppression agent is an RNAi molecule.

3. The method of claim 2, wherein the gene suppression agent is an miRNA.

4. The method of claim 1, wherein the gene suppression agent is a CRISPR system.

5. The method of claim 1, wherein the gene suppression agent and the exogenous wild-type allele are administered simultaneously in a single vector.

6. The method of claim 1, wherein the gene suppression agent and the exogenous wild-type allele are administered separately in two vectors.

7. The method of claim 1, wherein the endogenous gene causing the hearing loss is an exon listed in Table 1, Table 2, or is ACTG1, CCDC50, CEACAM1, COCH, COL11A2, CRYM, DENA5, DIABLO, DIAPH1, DSPP, EYA4, GJB2, GJB3, GJB6, GRHL2, HOMER2, KCNQ4, MYH14, MYH9, MYO1A, MYO6, P2RX, POU4F3, SLC1748, TBC1D24, TECTA, TJP2, TMC1, TNC, or WFS1.

8. (canceled)

9. A method of treating genetic hearing loss (GHL) in a patient in need thereof comprising:
(a) identifying a mutation in a GHL-causing gene, wherein the mutation causes GHL in the patient, and wherein the GHL-causing gene is an exon listed in Table 1; and
(b) administering to the patient a pharmaceutical composition comprising a therapeutic miRNA and a pharmaceutically acceptable carrier, wherein the GHL therapeutic miRNA is of 18 to 25 nucleotides in length and knocks-down the GHL-causing gene function at a higher level than it knocks-down gene function in a corresponding wild-type gene.

10. The method of claim 8, wherein the miRNA is 20 to 22 nucleotides in length.

11. (canceled)

12. The method of claim 9, wherein the miRNA knocks-down the GHL-causing gene function by at least 50% more than it knocks-down the corresponding wild-type gene function.

13. (canceled)

14. The method of claim 9, wherein the miRNA is contained in an expression cassette comprising a promoter operably linked to a nucleic acid encoding the miRNA.

15-18. (canceled)

19. The method of claim 14, wherein the expression cassette further comprises a marker gene.

20. (canceled)

21. The method of claim 14, wherein the expression cassette is contained in a vector.

22. The method of claim 21, wherein the vector is an adeno-associated virus (AAV) vector or an adenovirus vector.

23. The method of claim 9, wherein the pharmaceutical composition is administered intravenously and/or directly into the patient's inner ear.

24. A method of transducing cochlear epithelial tissue in an animal, comprising administering rAAV comprising a therapeutic agent to the animal,
wherein the administration is intravenous and the rAAV crosses the blood-labyrinthine barrier in the animal, and wherein the rAAV transfects spiral ganglion neurons, inner hair cells, outer hair cells, stria vascularis, and/or vestibular organs.

25. The method of claim 24, wherein the therapeutic agent is an RNAi molecule.

26. The method of claim 24, wherein the RNAi molecule is an miRNA.

27. The method of claim 24, wherein the rAAV is rAAV2/9.

28. The method of claim 24, wherein the administration is intravenous by means of superficial temporal vein in the animal.

29-34. (canceled)


SEQUENCE LISTING (10 sequences)

SEQ ID NO: 1 - RNA, 21 nt, synthetic construct
  misc_feature 1..21: note = Description of Artificial Sequence: Synthetic oligonucleotide
  source 1..21: mol_type = other RNA
gacgatcatgtacaggaccgg

SEQ ID NO: 2 - DNA, 21 bp, synthetic construct
  misc_feature 1..21: note = Description of Artificial Sequence: Synthetic oligonucleotide
  source 1..21: mol_type = other DNA
ccggtcctgtacatgatcgtc

SEQ ID NO: 3 - DNA, 21 bp, synthetic construct
  misc_feature 1..21: note = Description of Artificial Sequence: Synthetic oligonucleotide
  source 1..21: mol_type = other DNA
gacgatcatgtacaggaccgg

SEQ ID NO: 4 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  CDS 1..21: protein_id = 9; translation = PVLYMIV
  modified_base order(14,17,20): mod_base = OTHER; note = Uracil
ccggtcctgtacatgatcgtc

SEQ ID NO: 5 - protein, 7 aa, synthetic construct
  REGION 1..7: note = Description of Artificial Sequence: Synthetic peptide
PVLYMIV

SEQ ID NO: 6 - DNA, 21 bp, synthetic construct
  misc_feature 1..21: note = Description of Artificial Sequence: Synthetic oligonucleotide
  source 1..21: mol_type = other DNA
cactatcatatatagtactgg

SEQ ID NO: 7 - RNA, 21 nt, synthetic construct
  misc_feature 1..21: note = Description of Artificial Sequence: Synthetic oligonucleotide
  source 1..21: mol_type = other RNA
  CDS 1..21: protein_id = 10; translation = PVLYMIV
ccagtactatatatgatagtg

SEQ ID NO: 8 - RNA, 21 nt, synthetic construct
  misc_feature 1..21: note = Description of Artificial Sequence: Synthetic oligonucleotide
  source 1..21: mol_type = other RNA
gtgatagtatatatcatgacc

SEQ ID NO: 9 - protein, 7 aa, synthetic construct
PVLYMIV

SEQ ID NO: 10 - protein, 7 aa, synthetic construct
PVLYMIV